(19) United States
(12) Patent Application Publication
(10) Pub. No.: US 2018/0277250 A1
(43) Pub. Date: Sep. 27, 2018

(54) METHODS OF CHARACTERIZING AND/OR PREDICTING RISK ASSOCIATED WITH A BIOLOGICAL SAMPLE USING THERMAL STABILITY PROFILES

(71) Applicant: UNIVERSITY OF LOUISVILLE RESEARCH FOUNDATION, INC., Louisville, KY (US)

(72) Inventors: Nichola C. GARBETT, Louisville, KY (US); Guy N. BROCK, Worthington, OH (US)

(21) Appl. No.: 15/764,458

(22) PCT Filed: Oct. 17, 2016
(86) PCT No.: PCT/US16/57416

§ 371 Date: Mar. 29, 2018

Related U.S. Application Data
(60) Provisional application No. 62/241,819, filed on Oct. 15, 2015.

(51) Int. Cl. G16H 50/30; G06N 99/00; G01N 25/20; G01N 25/00

(52) U.S. Cl. CPC G16H 50/30; G06N 99/005; G01N 2800/52; G01N 25/005; G01N 2800/104; G01N 25/20

(57) ABSTRACT

Provided are methods of characterizing and/or predicting risk associated with a biological sample using thermal stability profiles. The methods include obtaining a thermal stability profile of the sample, using a sensor which detects heat capacity values, applying a classification algorithm to the thermal stability profile, and comparing the results thermal stability data in the database to characterize and/or predict risk of the condition. The methods may additionally include a classification algorithm selected from one or more of logistic regression, support vector machines, Fisher's linear discriminant analysis, modified version of Fisher's linear discriminant analysis (MLDA), and partial least squares.

### RELATED APPLICATIONS

This application claims the benefit of U.S. Provisional Application Ser. No. 62/241,819, filed Oct. 15, 2015, the entire disclosure of which is incorporated herein by this reference.

### GOVERNMENT INTEREST

This invention was made with government support under grant numbers P20RR018733, P20GM103482, and R21CA187345 awarded by the National Institutes of Health. The government has certain rights in the invention.

### TECHNICAL FIELD AND SUMMARY

The presently-disclosed subject matter generally relates to methods and systems for monitoring, predicting and/or identifying a likelihood of disease state in a subject. In particular, certain embodiments of the presently-disclosed subject matter relate to systems and methods making use of differential scanning calorimetry results and patterns for use in monitoring, predicting and/or identifying samples associated with a predictive outcome or status, including in some embodiments, a predictive outcome and/or status associated with a condition or disease. Embodiments of the systems and methods make use of algorithms, equipment, data patterns, and other tools as disclosed herein.

### BACKGROUND

According to the Autoimmune Diseases Coordinating Committee, as many as 24 million people in the US are afflicted with autoimmune disease. Diagnosis of autoimmune disease is particularly difficult because of highly diverse clinical manifestations. Systemic lupus erythematosus (SLE), a prototypic autoimmune disease that is heterogeneous in its presentation, is diagnosed based on clinical history, physical exam and laboratory studies including serological markers. Serological markers are problematic since the ones that are most sensitive are the least specific. This makes early diagnosis difficult and can result in a delay in important treatment. There has been a significant push to develop biomarkers that can accurately establish a diagnosis of SLE, evaluate disease activity, predict prognosis and guide therapy. Despite some promising studies deserving of further attention, few have been validated to-date. New diagnostic approaches are therefore of critical importance for both diagnosis and monitoring of SLE and SLE-related disease.

One potential source of multi-purpose diagnostic biomarkers is differential scanning calorimetry (DSC) profiles (or thermograms). Thermograms indicate the heat change (excess specific heat capacity) in a fluid sample as it is heated, corresponding to the structural changes in the molecular constituents of the fluid as a function of temperature (e.g., protein denaturation). DSC thermograms have been successfully used as a diagnostic tool for the characterization of human diseases, including cervical cancer, breast cancer, colorectal cancer, multiple myeloma, brain tumors, chronic obstructive pulmonary disease, and early renal function decline in type 1 diabetes patients. Most relevantly, Garbett et al. previously illustrated differences between average thermograms in a small sample of healthy controls, SLE patients, rheumatoid arthritis (RA) patients, and Lyme disease patients. Fish et al. extended these findings to a sample of 300 SLE patients and 300 healthy controls, demonstrating that thermograms could classify SLE patients versus healthy controls with similar accuracy to that based on immunological based markers. However, none of the aforementioned studies developed approaches for applying thermograms to enhance current diagnostic approaches for a given disease. Further, few of the studies have reported on the potential heterogeneity of thermograms along important demographic, clinical and environmental factors.

To facilitate interpretation of DSC data of clinical samples, a number of studies have reported the calculation of metrics that provide a read-out of specific localized features of DSC profiles (e.g. heat capacity and temperature maxima of profiles). These features have been useful in discerning trends in clinical groups and calculating the statistical significance of these differences. To utilize information from the entire thermogram for diagnostic classification a number of global analysis methods have been developed. One approach used a non-parametric method to determine differences between DSC profiles based on the distance between a test profile and averaged profiles for each class. The distance was defined as the geometric average of the correlation between DSC profiles (i.e. similarity in shapes) and Euclidean distance. This approach was used to analyse DSC profiles of healthy controls and lupus patients and achieved 82% correct classification of healthy profiles and 88% for lupus. The method is generally applicable to other data sets where any test profile can be compared to a well-defined reference group.

Other groups have applied this approach for the analysis of DSC data in different settings, for example, for the classification of colorectal cancer based on DSC profiles. Another method for the analysis of plasma profiles employed a parametric statistical model developed for the classification of cervical cancer versus healthy controls. Here, DSC profiles were reduced in complexity by restricting the temperature range to that encompassing the major heat capacity signal (50-76° C.) and averaging over 1° C. temperature increments. Profiles were then subjected to a logarithmic transformation and fit to a linear regression model. This method performed extremely well for the healthy/cervical cancer data set with a mean classification rate of 97%. As the model used in this approach was developed from this specific data set future development would require the evaluation of this approach with other data sets. Another useful method was based on deconvoluting the DSC profile into several component curves each with a defined height, center and width which were used in a multiparametric analysis for the classification of healthy controls and gastric adenocarcinoma patients. The construction of polygonal plots from these three parameters for each of the component curves provided a useful graphical tool to distinguish patient groups. Also, similar to some earlier reports, the area and first moment, or average, temperature of DSC profiles were found to display differences between the controls and gastric adenocarcinoma patients.

The approaches discussed above demonstrate the evolution in the development of analytical tools for the characterization of DSC biofluid profile features associated with various clinical conditions. However, there remains a desire for the development and validation of a reliable analysis approaches to provide a rapid, easily interpretable diagnostic result that can be readily employed in the clinical setting.

### SUMMARY

The presently-disclosed subject matter meets some or all of the above-identified needs, as will become evident to those of ordinary skill in the art after a study of information provided in this document.

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned, likewise, those features can be applied to other embodiments of the presently disclosed subject matter, whether listed in this Summary or not. To avoid excessive repetition, this Summary does not list or suggest all possible combinations of such features.

In some embodiments, the presently-disclosed subject matter includes a method of characterizing and/or predicting risk of a condition associated with a biological sample obtained from a subject, the method including obtaining a thermal stability profile of the sample using a sensor which detects heat capacity values, applying a classification algorithm to the thermal stability profile, and comparing the results to thermal stability data in the database to characterize and/or predict risk of the condition. In one embodiment, the condition is systemic lupus erythematosus (SLE). In another embodiment, the method includes classifying the subject as having SLE when at least four of eleven ACR SLE criteria are present in the biological sample. Additionally or alternatively, the method may include treating the subject for the condition.

In certain embodiments, the classification algorithm is selected from one or more of logistic regression, support vector machines, Fisher's linear discriminant analysis, modified version of Fisher's linear discriminant analysis (MLDA), and partial least squares. For example, in one embodiment, the classification algorithm includes a modified version of Fisher's linear discriminant analysis (MLDA). In another embodiment, the method also includes a serological based classification, the MLDA and the serological based classification together providing increased sensitivity and overall accuracy for systemic lupus erythematosus (SLE) patients versus controls. In a further embodiment, the method includes characterizing the subject as having systemic lupus erythematosus (SLE) that is not detectable through antibody testing.

The sensor includes any suitable sensor, such as, but not limited to, a differential scanning calorimeter (DSC). In some embodiments, the method also includes characterizing the thermogram by one or more metrics selected from: (1) the total area under the thermogram (optionally from 45-90° C.); (2) the maximum excess specific heat capacity at various peaks; (3) the overall maximum peak height; (4) the width of the primary thermogram peak at half height; (5) the temperature of the peak maximum (Tₘₐₓ); (6) the ratio of the peak heights; and (7) the “mean” or first moment temperature of the thermogram, T_{FM}, where

$${T}_{FM} = \frac{{\int }_{45}^{90} ( {TC}_{p}^{ex} ) dT}{{\int }_{45}^{90} {C}_{p}^{ex} dT}$$

and C_{F}^{ex} represents the excess specific heat capacity at a given temperature.

The presently-disclosed subject matter also includes a method of characterizing and/or predicting risk of a condition associated with biological sample obtained from a subject, comprising obtaining a thermogram of the sample using a sensor which detects heat capacity values, analyzing the thermogram using localized thermogram features and principal components, and comparing the results to data in a database to characterize and/or predict risk of the condition. In some embodiments, the method also includes applying a classification algorithm to the thermal stability profile.

Embodiments of the presently disclosed subject matter further include a method of characterizing and/or predicting risk of a condition associated with biological sample obtained from a subject, comprising obtaining a thermogram of the sample using a sensor which detects heat capacity values, characterizing the thermogram by one or more metrics selected from: (1) the total area under the thermogram (optionally from 45-90° C.); (2) the maximum excess specific heat capacity at various peaks; (3) the overall maximum peak height; (4) the width of the primary thermogram peak at half height; (5) the temperature of the peak maximum (Tₘₐₓ); (6) the ratio of the peak heights; and (7) the “mean” or first moment temperature of the thermogram, T_{FM}, where

$${T}_{FM} = \frac{{\int }_{45}^{90} ( {TC}_{p}^{ex} ) dT}{{\int }_{45}^{90} {C}_{p}^{ex} dT}$$

and C_{P}^{ex} represents the excess specific heat capacity at a given temperature, and comparing the results to data in a database to characterize and/or predict risk of the condition.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1. Median thermogram and principal component data for lupus and control subjects. (Left panel) Solid lines represent median thermogram values for lupus and control subjects at each temperature. Confidence bands represent 10^{th} and 90^{th} percentiles for each group of subjects. (Right panel) Loadings of the first four principal components for the thermogram data (combined lupus and control samples).

FIG. 2. Scatter plot matrix for thermogram peak metrics. Scatter plots are constructed for each pairwise combination of the excess specific heat capacity (cal/° C.g) for the three prominent peaks in the thermogram data. Points are color-coded according to lupus/control status.

FIG. 3. Scatter plot matrix for selected thermogram metrics. Scatter plots are constructed for each pairwise combination of the temperature of the peak maximum (Tₘₐₓ), first moment temperature (T_{FM}), and ratio of C_{P}^{ex} at Peak 1 to C_{F}^{ex} at Peak 3. Points are color-coded according to lupus/control status.

FIG. 4. ROC curves and area under the ROC curve (AUC) values based on the first six principal components of the lupus thermogram data.

FIG. 5. ROC curves and area under the ROC curve (AUC) values for six of the calculated summary metrics of the lupus thermogram data.

FIG. 6. Accuracy of the six evaluated classification methods for the lupus thermogram data. Box plots represent values from 100 test data sets created by splitting the data randomly into training (two thirds) and testing (one third) sets.

FIG. 7. Solution vectors for the six classification methods applied to the lupus thermogram data. In each case the blue line represents the median coefficient value for each thermogram temperature across the 100 training data sets, while the green shaded region represents 10^{th} and 90^{th} percentiles from the 100 training data sets. Training data sets were created by randomly splitting the data into training (two thirds) and testing (one third) sets.

FIG. 8: Plot of the median thermogram value at each temperature for lupus and control subjects along with bands representing the 5th and 95th percentiles among subjects at each temperature. The loadings for the first principal component among all subjects are shown as the black line.

FIG. 9: Plot of the median thermogram value at each temperature for lupus and osteoarthritis patients along with bands representing the 5th and 95th percentiles among subjects at each temperature. The loadings for the first principal component among all subjects are shown as the black line.

FIG. 10: Plot of the median thermogram value at each temperature for lupus and rheumatoid arthritis patients along with bands representing the 5th and 95th percentiles among subjects at each temperature. The loadings for the first principal component among all subjects are shown as the black line.

FIG. 11: Scree plot for principal components of DSC thermograms based on all subjects (lupus patients and controls).

FIG. 12: Boxplots of summary statistics calculated for thermograms of lupus patients and controls. Top Row (from left to right): Total area under the curve, width at half height, and height at maximum temperature. Middle Row: Excess specific heat capacity (C_{P}^{ex}) at Peak 1 (62-67° C.), Peak 2 (69-73° C.), and Peak 3 (75-80° C.). Bottom Row: Temperature at the maximum peak (Tₘₐₓ), first moment temperature (T_{FM}), and ratio of C_{F}^{ex} at Peak 1 to C_{P}^{ex} at Peak 2.

FIG. 13: Density of temperature at maximum peak thermogram height (Tₘₐₓ) for controls and lupus patients. The density plots reveal roughly three prominent peaks among the subjects at 62-67° C., 69-73° C., and 75-80° C. (the latter being present only among lupus patients).

FIG. 14: Plot of the median thermogram value at each temperature for lupus and control subjects stratified by gender and ethnicity. Bands represent the 5th and 95th percentiles among subjects at each temperature.

FIG. 15: Plot of the median thermogram value at each temperature for lupus and control subjects stratified by presence/absence of anemia (not applicable indicates that the study question did not apply). Bands represent the 5th and 95th percentiles among subjects at each temperature.

FIG. 16: Plot of the median thermogram value at each temperature for lupus and control patients stratified by level of Anti-Cardiolipin Immunoglobulin G (cut-point at the median value of 6). Bands represent the 5th and 95th percentiles among subjects at each temperature.

FIG. 17: Sensitivity, specificity, and overall accuracy for classifying lupus patients vs. controls based on DSC thermograms only (DSC), antibody tests only (Ab), and combined DSC/antibody tests (DSC+Ab). Boxplots represent values from 1000 test data sets created by splitting the data randomly into training (two thirds) and testing (one third) sets.

FIG. 18: Sensitivity, specificity, and overall accuracy for classifying lupus patients vs. osteoarthritis patients based on DSC thermograms only (DSC), antibody tests only (Ab), and combined DSC/antibody tests (DSC+Ab). Boxplots represent values from 1000 test data sets created by splitting the data randomly into training (two thirds) and testing (one third) sets.

FIG. 19: Sensitivity, specificity, and overall accuracy for classifying lupus patients vs. rheumatoid arthritis patients based on DSC thermograms only (DSC), antibody tests only (Ab), and combined DSC/antibody tests (DSC+Ab). Boxplots represent values from 1000 test data sets created by splitting the data randomly into training (two thirds) and testing (one third) sets.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter generally relates to methods and systems for monitoring, predicting and/or identifying a likelihood of disease state in a subject. In particular, certain embodiments of the presently-disclosed subject matter relate to systems and methods making use of differential scanning calorimetry results and patterns for use in monitoring, predicting and/or identifying samples associated with a predictive outcome or status, including in some embodiments, a predictive outcome and/or status associated with a condition or disease. Embodiments of the systems and methods make use of algorithms, equipment, data patterns, and other tools as disclosed herein.

In some embodiments, the methods and systems disclosed herein use thermograms as a diagnostic tool in SLE. For example, in one embodiment, the methods and systems apply a classification algorithm to thermograms based on plasma samples from SLE patients and healthy controls. The SLE patients and healthy controls may be provided from any suitable source, such as, but not limited to, the Lupus Family Registry and Repository (LFRR). In another embodiment, the plasma samples include 300 SLE patients and 300 healthy controls from the LFRR. In a further embodiment, a comprehensive exploratory investigation of the heterogeneity among thermograms from SLE patients and healthy controls is provided, including stratification by important demographic variables, laboratory measurements, and environmental exposures. Still further, in certain embodiments, thermograms are combined with SLE immunological markers to improve upon classification based on the serological markers alone.

In some embodiments, the presently disclosed subject matter includes a method of characterizing and/or predicting risk of a condition associated with biological sample obtained from a subject, which involves obtaining a thermal stability profile of the sample, using a sensor which detects heat capacity values; applying a classification algorithm to the thermal stability profile; and comparing the results to thermal stability data in the database to characterize and/or predict risk of the condition. The sample includes any sample suitable for obtaining a thermal stability profile therefrom, such as, but not limited to, a plasma sample.

In some embodiments, the presently disclosed subject matter includes a method of characterizing and/or predicting risk of a condition associated with biological sample obtained from a subject, which involves obtaining a thermogram of the sample, using a sensor which detects heat capacity values; analyzing the thermogram using localized thermogram features and principal components; and comparing the results to data in a database to characterize and/or predict risk of the condition. The method can also involve applying a classification algorithm to the thermal stability profile. In some embodiments, the classification algorithm is selected from one or more of logistic regression, support vector machines, Fisher's linear discriminant analysis, partial least squares. The method can also involve further comprising characterizing the thermogram by one or more metrics selected from: (1) the total area under the thermogram (optionally from 45-90° C.); (2) the maximum excess specific heat capacity at various peaks; (3) the overall maximum peak height; (4) the width of the primary thermogram peak at half height; (5) the temperature of the peak maximum (Tₘₐₓ); (6) the ratio of the peak heights; and (7) the “mean” or first moment temperature of the thermogram, T_{FM}, where

$${T}_{FM} = \frac{{\int }_{45}^{90} ( {TC}_{p}^{ex} ) dT}{{\int }_{45}^{90} {C}_{p}^{ex} dT}$$

and C_{F}^{ex} represents the excess specific heat capacity at a given temperature.

In some embodiments, the presently disclosed subject matter includes a method of characterizing and/or predicting risk of a condition associated with biological sample obtained from a subject, which involves obtaining a thermogram of the sample, using a sensor which detects heat capacity values; characterizing the thermogram by one or more metrics selected from: (1) the total area under the thermogram (optionally from 45-90° C.); (2) the maximum excess specific heat capacity at various peaks; (3) the overall maximum peak height; (4) the width of the primary thermogram peak at half height; (5) the temperature of the peak maximum (Tₘₐₓ); (6) the ratio of the peak heights; and (7) the “mean” or first moment temperature of the thermogram, T_{FM}, where

$${T}_{FM} = \frac{{\int }_{45}^{90} ( {TC}_{p}^{ex} ) dT}{{\int }_{45}^{90} {C}_{p}^{ex} dT}$$

and C_{F}^{ex} represents the excess specific heat capacity at a given temperature; and comparing the results to data in a database to characterize and/or predict risk of the condition.

In some embodiments of the methods disclosed herein, the sensor comprises a differential scanning calorimeter (DSC).

Some embodiments of the methods disclosed herein also involve administering treatment to the subject. For example, in one embodiment, the methods include characterizing and/or predicting risk of a condition associated with a biological sample obtained from a subject, and treating the subject based upon the characterization and/or prediction. In another embodiment, characterizing and/or predicting risk of a condition includes classifying a subject as having SLE when at least four of eleven ACR SLE criteria are detected in the sample. In a further embodiment, characterizing and/or predicting risk of a condition includes a combination of thermograms and serological based classification, which increases sensitivity and/or overall accuracy for SLE patients versus controls.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter is further illustrated by the following specific but non-limiting examples. The following examples may include compilations of data that are representative of data gathered at various times during the course of development and experimentation related to the present invention.

### EXAMPLES

### Example 1

Materials and Methods.

Plasma Samples.

De-identified plasma samples and patient data were obtained from the Lupus Family Registry and Repository (LFRR). Plasma samples for 300 patients meeting the revised criteria of the American College of Rheumatology for SLE and 300 healthy controls matched demographically by sex, ethnicity and age were received and kept at −80° C. until thawed for DSC analysis.

Collection of DSC Thermograms.

DSC samples were prepared and analyzed according to our previously published procedure which includes a detailed account of our experimental procedures. Data were collected using an automated VP-Capillary DSC system (MicroCal, LLC, Northampton, Mass., now a division of Malvern Instruments Inc.). Electrical calibration of the differential power signal and temperature calibration using hydrocarbon temperature standards were performed as part of the manufacturer periodic instrument maintenance. Interim instrument performance was assessed using biological standards lysozyme and RNaseA. Samples and dialysate were loaded into 96 well plates thermostated at 5° C. within the instrument autosampler until analysis. Thermograms were recorded from 20° C. to 110° C. at a scan rate of 1° C./min with a pre-scan thermostat of 15 minutes, mid feedback mode and a filtering period of 2 seconds. Duplicate thermograms were obtained for each plasma sample. DSC data were analyzed using Origin 7 (OriginLab Corporation, Northampton, Mass.). Raw DSC data were corrected for the instrumental baseline by subtraction of a suitable buffer reference scan. Thermograms were normalized for the total protein concentration and corrected for non-zero baselines by application of a linear baseline fit. Final thermograms were plotted as excess specific heat capacity (cal/° C.g) versus temperature (° C.).

Summary Metrics of DSC Thermograms.

Thermograms are frequently characterized by metrics summarizing the shape and prominent features of the thermograms. These include: (1) the total area under the thermogram (typically from 45-90° C.); (2) the maximum excess specific heat capacity at various peaks (e.g. Peak 1 height, Peak 2 height, etc.); (3) the overall maximum peak height; (4) the width of the primary thermogram peak at half height; (5) the temperature of the peak maximum (Tₘₐₓ); (6) the ratio of the peak heights (e.g., (Peak 1 height)/(Peak 2 height), etc.); and (7) the “mean” or first moment temperature of the thermogram, T_{FM}, where

$${T}_{FM} = \frac{{\int }_{45}^{90} ( {TC}_{p}^{ex} ) dT}{{\int }_{45}^{90} {C}_{p}^{ex} dT}$$

and C_{F}^{ex} represents the excess specific heat capacity at a given temperature. These summary metrics can be used in lieu of the original thermogram values for classifying disease status based on any of the classification models described below. While the calculated metrics are useful for characterizing certain aspects of the thermograms, they are not necessarily informative for differences between patient classes.

Principal components (PCs) are another common technique for summarizing the information in a data matrix in a concise manner. PCs are not intended as a classification technique per se, but are commonly used as a dimension reducing tool prior to building a classification or regression model. PCs are the set of orthogonal vectors or factors such that the first vector is the direction which explains the most variation in the data, the second vector is the direction which explains the second greatest percentage of variation in the data that is orthogonal to the first, and so on. The solution can be obtained from the eigenvalue decomposition of the covariance matrix of the data, where the principal components directions are the eigenvectors of the covariance matrix and the variance of the principal components are proportional to the eigenvalues of the covariance matrix. Typically, only the number of components needed to explain 90-95% of the total variation in the data are retained (e.g., as determined by the “elbow” in a scree plot). Once the principal components are determined, these can be used as a replacement for the original variables in a classification problem. The PCs have the advantage that they are orthogonal and hence avoid computational issues associated with multicollinearity. However, they are not specifically designed for a classification problem and do not make explicit use of the clinical classification of the data in their construction. Hence they are frequently sub-optimal for classification problems.

Classification Methods.

The thermogram values at each temperature can be treated as variables and used to develop classification models for diseased versus healthy individuals. Since the number of thermogram values is large and can potentially lead to overfitting, variable selection techniques should be employed to reduce the dimension of the problem. This can be accomplished via sparse or penalized methods, which are typically available for the more commonly used classification methods. The goal is to use the information in the thermogram profiles to classify a patient as having a disease (here, lupus) or not. That is, to develop a phenomenological model governed by a set of parameters that can be used to predict the class label (i.e. disease or not) of each thermogram. The unconstrained solutions to the problem (i.e., the parameter estimates associated with each of the predictor variables) are typically obtained by minimizing a suitable objective function (e.g., for statistical models this is typically the negative log-likelihood function). The idea behind penalized methods is to employ a penalty in the objective function which prevents overly-complex solutions. These penalty functions are based on the magnitude of the coefficient vector, and can either shrink the coefficients overall (the ridge or L₂ penalty) or eliminate some of the coefficients entirely (the ‘least absolute shrinkage and selection operator’ (lasso) or L₁ penalty). The latter penalty function is a form of variable selection since some of the coefficients are shrunk to zero (and thus eliminated from the model). Other possibilities for penalty functions exist, including the elastic net which is a compromise combining the ridge and lasso penalty functions and tends to retain or discard groups of correlated variables together. The degree of penalization is controlled by a parameter which varies from very stringent (e.g., all parameters are shrunk to zero) to nonexistent (the unconstrained solution). The optimal level of shrinkage or penalization is usually determined empirically by a cross-validation process. A good introduction to penalized methods for classification is available in Hastie, Tibshirani, and Friedman, especially Chapter 18. Below is a brief description of the classification methods and software packages that were used for analyzing the DSC thermograms in this study.

### Logistic Regression (LR)

Logistic regression is an example of a generalized linear model (GLM), which extends the statistical theory for linear models to the case where the response variable (here, heat capacity) is non-normally distributed. In this context the logistic regression model models the probability that an individual will have lupus, given the set of input thermogram values at specific temperatures. Predicted probabilities can be obtained from the model and used to classify patients as having lupus or not (e.g., using a threshold probability of 0.5). Penalized solutions to the problem are available in a number of R packages including lqa and glmnet. The glmnet package uses the elastic net penalty and allows users to select between the lasso, ridge, or any weighted combination of the two penalties. In this work we use the glmnet package with both the lasso (LR-LASSO) and elastic net (LR-ENET, equally weighted combination of lasso and ridge) penalties.

### Support Vector Machines (SVM)

Support vector machines have enjoyed great success in classification problems since their introduction. The idea behind SVMs is to find the hyperplane in multidimensional space such that the margin (or separation) between the training points for the two classes is maximized. While SVMS do not return a predicted probability, historically they have been very successful for classification problems. Several potential advantages of SVMs include the focus on points (subjects) which characterize the boundary between two classes and the ability to incorporate features mapped into a different space via a kernel function (a function which computes the similarity or proximity of two points in the transformed space). In this work we use the penalizedS VM package in R to fit penalized SVM models using a linear kernel. In addition to the lasso penalty, the package also implements the Smoothly Clipped Absolute Deviation (SCAD) penalty. The SCAD penalty behaves similarly to the lasso for small coefficients but retains the large coefficients as they are. We evaluate both the SCAD penalized SVM model (SVM-SCAD) and the SCAD penalty combined with an L₂ penalty, the elastic SCAD (SVM-ESCAD).

### Fisher's Linear Discriminant Analysis (LDA)

The goal of Fisher's LDA is to find the direction in the covariate space that best separates the two (or more) classes of patients. That is, the linear combination of the covariates which maximizes the ratio of the between group variation to the within group variation. When the number of covariates (here, heat capacity values at each temperature) far exceeds the number of subjects, classical LDA cannot be directly applied and a regularized or penalized approach is needed. This can be accomplished by shrinking the covariance matrix to the more stable and easily invertible identity matrix (a matrix consisting of ones on the diagonal and zeros elsewhere) and substituting this into the LDA algorithm. Other authors included an L₁ penalty or L₁ constraint on the objective function for LDA to enforce dimension reduction (sparsity) on the coefficients of the resulting discriminant vector. R packages which employ the latter approaches include penalizedLDA and MGSDA. In this work we evaluate the L₁ or lasso constrained LDA as estimated by the MGSDA package (LDA-LASSO).

Partial Least Squares (PLS)

Partial least squares is similar to principal components, but instead of finding orthogonal factors that explain the most variation in the covariates the components are determined which find the greatest covariance between the covariates and the response variable. In this case, the first PLS component is the linear combination of the thermogram values which has the strongest covariance with the disease status (lupus or normal), and subsequent components orthogonal to the first are determined in an analogous fashion. PLS has a long history of application in chemometrics (see [47] for a recent review of application within metabolomics data). Similarly to the above classifiers, penalized versions achieving a sparse coefficient or loading vectors are obtained by directly penalizing the objective function. When the response variable is binary, a version of PLS called PLS-DA (for PLS discriminant analysis) is applied. In the R package spls this is achieved by a two-step process where sparse PLS is first applied for dimension reduction (sparsity) and PLS dimensions are subsequently used in an LDA or logistic regression classifier. In this work we evaluate the sparse PLS-DA method as obtained by the splsda function in package spls (SPLS-DA).

### Results

To develop preliminary approaches for enhanced data analyses of DSC data we utilized our most substantial DSC data set collected using samples from the Lupus Family Registry and Repository (LFRR) which is comprised of 300 lupus patients and 300 demographically-matched controls. We examined an array of approaches to characterize thermogram differences related to clinical status in this large data set and determined the performance of each approach for the diagnostic classification of patients based on DSC data. Two case samples and six control samples were flagged as poor quality data and removed prior to analysis.

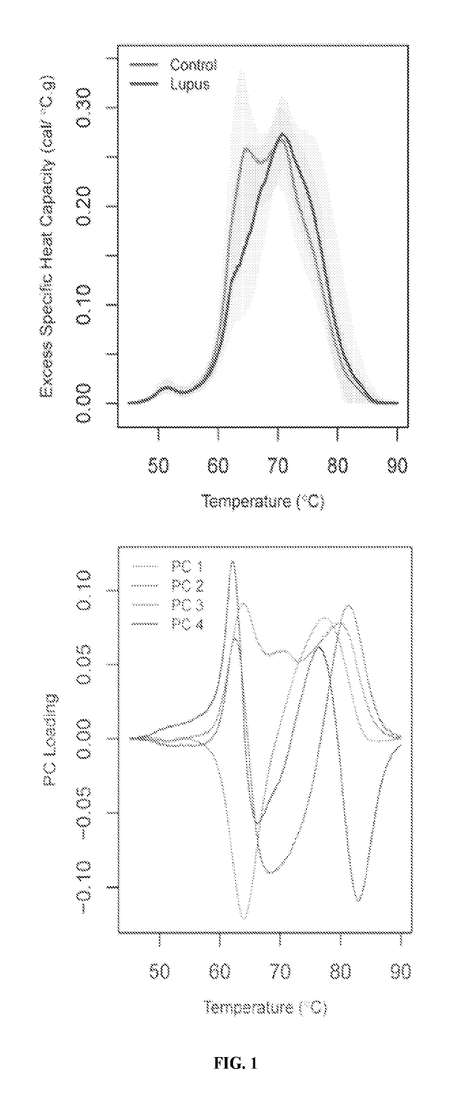

FIG. 1 (left panel) displays the median thermogram profiles for both lupus and control patients along with empirical 10^{th} and 90^{th} percentiles at each temperature. A casual inspection reveals prominent differences between the two profiles at the first peak (62-67° C.) and a third peak around 75-80° C. The second peak (69-73° C.) is more similar between the two groups, though still statistically significantly different (p<0.001, t-test). These visual findings are corroborated by the first principal component (PC) (FIG. 1, right panel), which contrasts differences between the heights of the first and third peaks. That is, subjects with larger values of peak 3 and smaller values of peak 1 (e.g., lupus patients) will also have larger values of the first PC. The second PC has all positive loadings and is associated with the total area under the thermogram curve. The third and fourth PCs are more difficult to interpret, but seemingly involve contrasting thermogram values primarily in the 60-62° C., 69-71° C., 74-76° C., and 80-82° C. ranges.

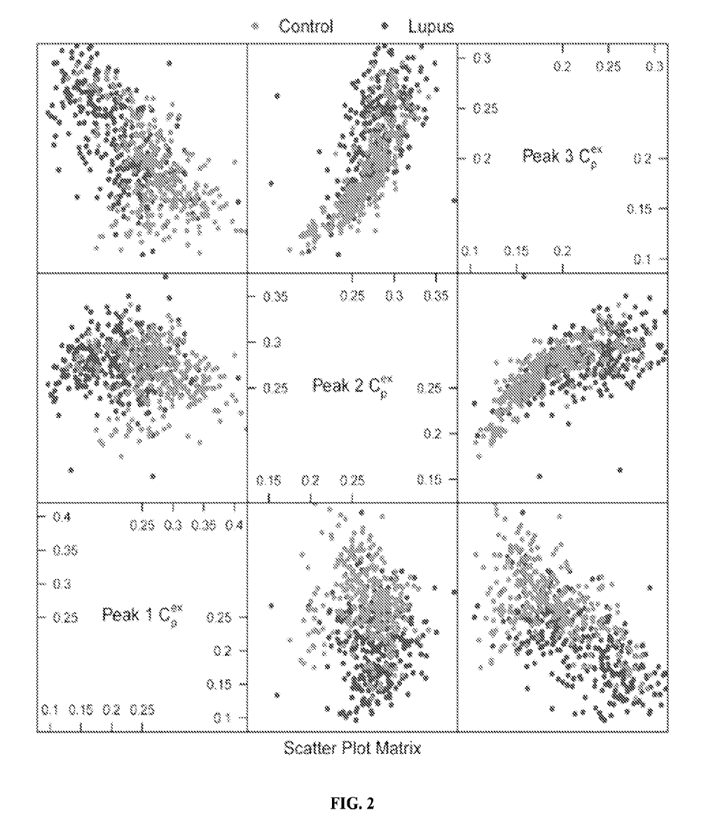

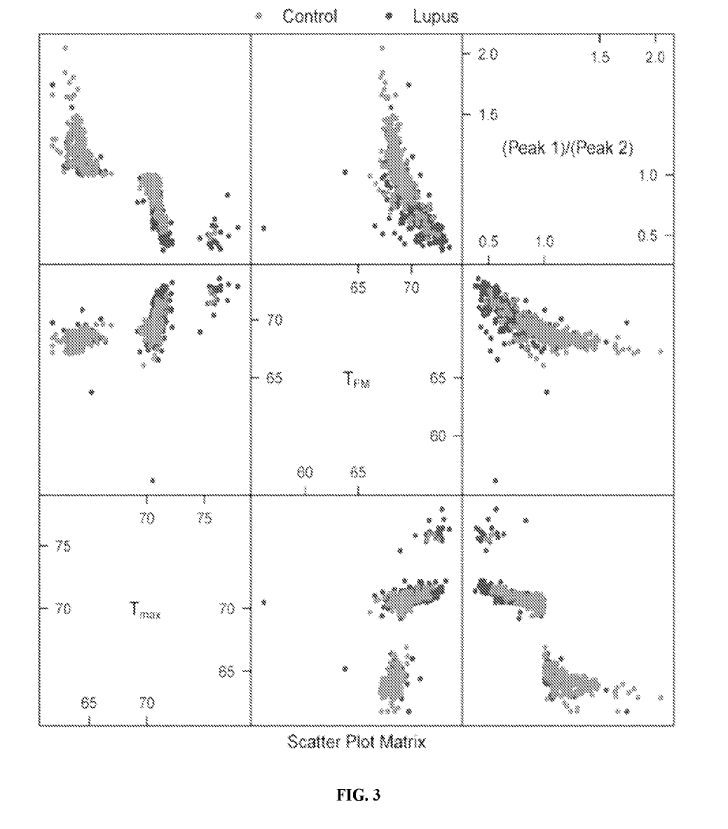

Scatter plot matrices for the maximum peak heights of peaks 1, 2, and 3 are shown in FIG. 2, color-coded by lupus or control status. The figure reiterates what was observed in FIG. 1, namely that peaks 1 and 3 differ between lupus patients and controls while peak 2 largely does not. However, there is considerable overlap between the two groups of subjects in the scatter plots. FIG. 3 similarly plots scatter plot matrices for Tₘₐₓ, T_{FM}, and the (peak 1)/(peak 3) ratio. While there are interesting distributional patterns only the (peak 1)/(peak 3) ratio offers any substantial separation between the groups. However, all the summary metrics were statistically significantly different (p<0.05, t-test) between lupus cases and controls with the exception of the width at half height.

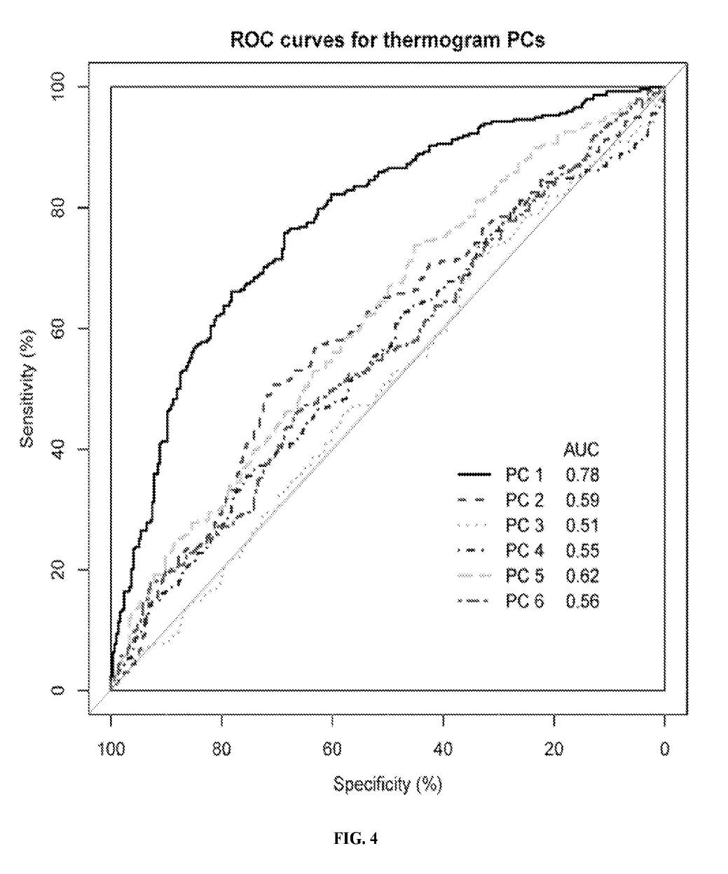

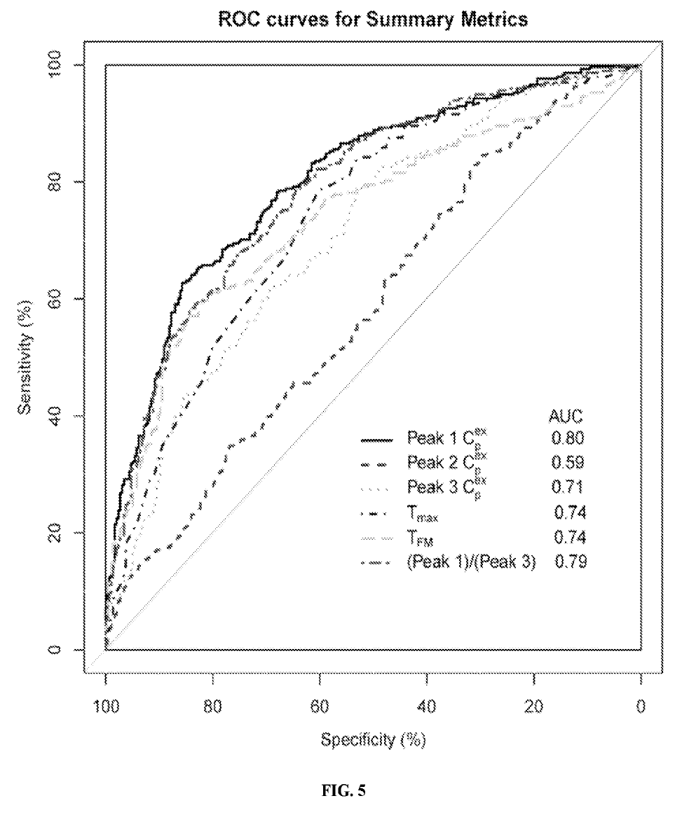

The utility of the summary metrics and PCs for patient classification can be further illustrated via receiver operating characteristic (ROC) curves. For each possible cut-point of the summary metrics/PCs, patients are classified as having lupus or not (e.g., based on whether they are above/below the cut-point). The sensitivity and specificity of the resulting classification is determined by comparing to the true disease status. ROC curves are then constructed by plotting the resulting sensitivity and one minus the specificity values for all of these cut-points. The area under the curve (AUC) gives an overall measure of predictive ability called the concordance index or C-index, with values of 0.5 corresponding to a random guess and values of 1.0 corresponding to perfect separation. FIG. 4 plots the ROC curves for the first six PCs, while FIG. 5 plots ROC curves for six of the calculated summary metrics. While the first PC is useful for separating patients (AUC=0.78), the remaining are not discriminatory with a maximum AUC value of 0.62. Likewise, peak 1 and the (peak 1)/(peak 3) ratio are useful discriminators (AUC 0.80) while peak 3, Tₘₐₓ and T_{FM} are useful to a lesser extent (AUC between 0.71 and 0.74) and peak 2 is not particularly predictive (AUC=0.59).

To evaluate the classification accuracy of models based on PCs and summary metrics we divided the subjects into a training set (⅔^{rds}) and a test set (remaining ⅓′¹). A logistic regression model was fitted using the first six PCs based on the training data, and likewise a similar model was fitted using five of the six summary metrics displayed in FIG. 5 (peak 1, peak 3, (peak 1)/(peak 3) ratio, Tₘₐₓ, and T_{FM}). The test data accuracy for the two models was very similar: 70.9% for the PC model and 70.4% for the summary metrics model.

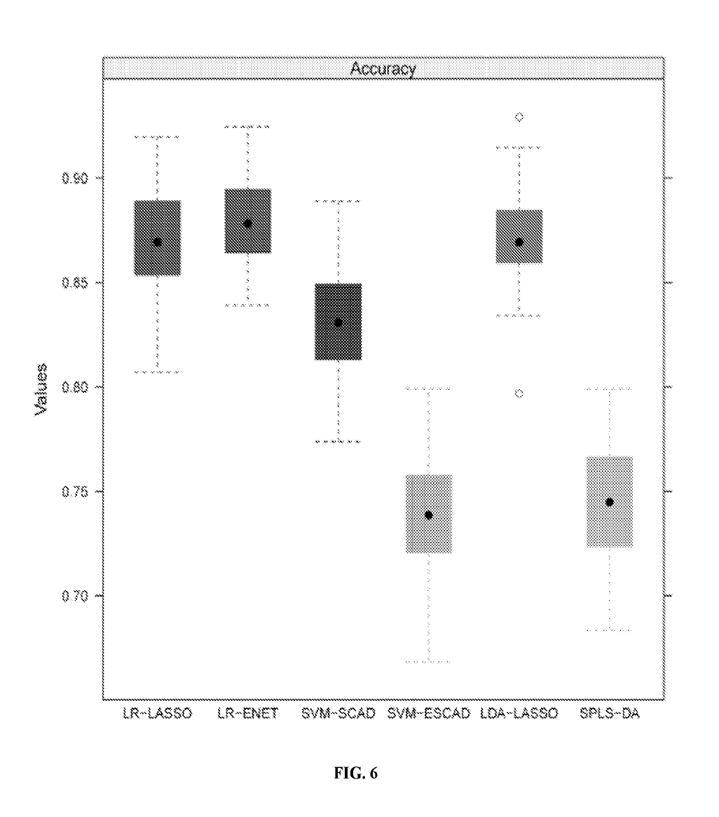

We subsequently evaluated the performance of the six classification models: LR-LASSO, LR-ENET, SVM-SCAD, SVM-ESCAD, LDA-LASSO, and SPLS-DA. To capture the most informative range of the thermogram the temperature was restricted to between 60.0 and 80.9° C. Since thermogram values were recorded in 0.1° C. increments, this resulted in 210 total features available for classification purposes. To assess variability in the classification accuracy and the solution vector of the methods we randomly split the data 100 times into a ⅔^{rds} training set and ⅓^{rd} test set. The overall accuracy for the six models on the 100 test data sets are displayed in FIG. 6. The best performing models were clearly LR-ENET (median accuracy 88%), LR-LASSO (87%), and LDA-LASSO (87%). This was followed by SVM-SCAD (83%), while SVM-ESCAD (74%) and SPLS-DA (74%) had the lowest test data accuracy levels. Note however that all of the models outperformed classification based on the PCs and summary metrics.

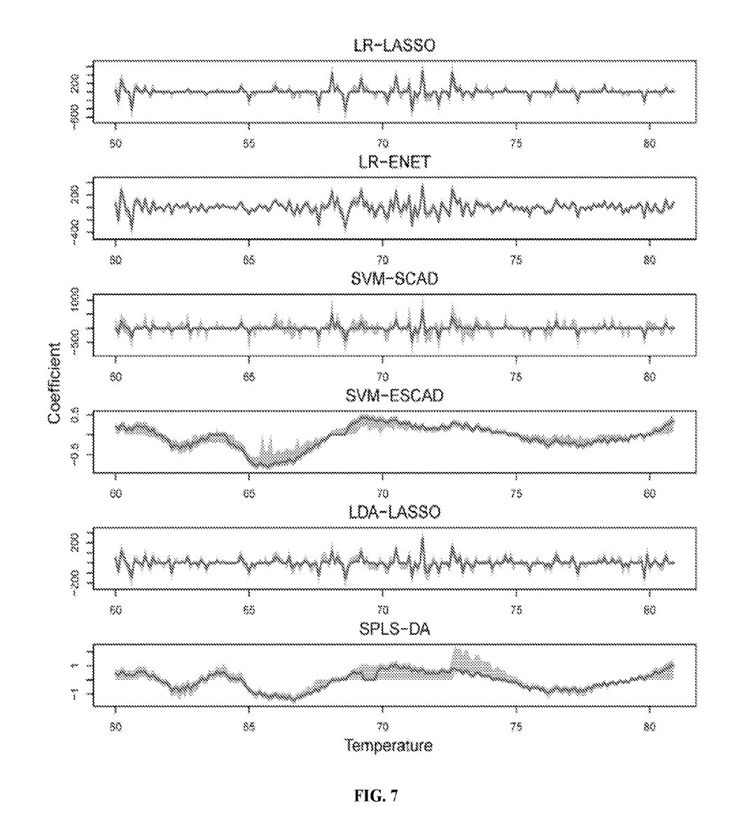

Since only two classes of subjects were being compared, in each case the solution for the classifier resulted in a single discriminatory variable calculated as a weighted average of the thermogram values. The weights correspond to the solution vector (e.g., the coefficient vector) and are plotted in FIG. 7 across the 100 splits of the data. Note that the dimension reduction (sparsity) criterion coincides with the zero coefficients in the solution, which is particularly evident in the lasso constrained solutions. Also, there is a remarkable degree of similarity in the solution pattern for LR-LASSO, LR-ENET, LDA-LASSO, and to a lesser extent SVM-SCAD. In particular, the pattern between 67 and 72.5° C. is consistently maintained across all the methods (the green shaded region in each of the plots gives the 10th and 90th percentiles for the solution vector across the 100 data splits). Patterns around 60° C., 65° C., 77° C., and 80° C. are also fairly well maintained. In contrast, the solution patterns for SVM-ESCAD and SPLS-DA are decidedly different from the other four classifiers and also similar to each other. While the solution for these two classifiers appears ‘smoother’ compared to the other four, the classification accuracy is notably lower (FIG. 6). A final note concerns the difference in magnitude of the coefficients between SVM-ESCAD/SPLS-DA and the other four classifiers. However, the large magnitude of the coefficients for these four classifiers does not result in high variability in classification accuracy, as evidenced by FIG. 6.

### Discussion

Describe above is the use of DSC thermograms as a diagnostic tool and illustrate its application for classifying lupus cases versus controls. We compared classification accuracy based on summary metrics of the thermograms with classification algorithms specifically tuned to distinguish lupus cases from controls based on the thermogram information. Penalized methods were used to constrain the solution and reduce the dimension of the problem. Our results indicate that substantially improved performance is obtained with the classification algorithms relative to summary metrics/PCs alone, particularly for LR-LASSO, LR-ENET, and LDA-LASSO.

In contrasting the results from the different classification algorithms, the solutions could be grouped into two sets of high similarity. While the coefficient vectors for SVM-ESCAD and SPLS-DA were fairly smooth and seemingly easier to interpret, the classification accuracies based on the LDA, SVM-SCAD, and LR methods were substantially better. Though at first glance the coefficient vectors obtained for LDA-LASSO, LR-ENET, LR-LASSSO, and SVM-SCAD appear ‘noisy’, the solutions were very similar to each other and relatively consistent across multiple splits of the data. Thus these coefficient patterns may relay important information concerning contrasting elements of the thermogram profiles that distinguish diseased (here, lupus) and healthy individuals. Theoretical connections between the loss functions (objective functions) for LDA, LR, and SVM are discussed in Section 12.3.2 in.

To handle the dimensionality of the problem, we used penalized methods to simultaneously obtain the solution vector and select important thermogram values for classification. Other options for variable selection include filter and wrapper methods. Filter methods involve selecting variables based on a univariate test statistic (e.g., a t-test or Wilcoxon test for differences between cases and controls) applied to each variable. The variables with the most significant results (usually based on a pre-defined p-value threshold) are then used for classification. While simple to apply, the combination of variables selected are not necessarily ideal for classification. In contrast, wrapper approaches are designed to select variables optimal for a particular classification algorithm. This is accomplished by defining subsets consisting of a decreasing number of variables, where for each subset the variables are ranked by a variable importance measure and the least significant predictors are removed to obtain the next subset. In order to avoid over-fitting, such approaches are typically wrapped within a double cross-validation scheme. Wrapper methods are thus more comparable to penalized approaches, but may be more computationally burdensome (depending on the coarseness of the number of subsets evaluated). The caret package in R is a good resource for applying many classification algorithms coupled with wrapper selection and variable importance measures.

The thermogram value at each 0.1° C. temperature increment were used as input to allow maximum flexibility for the classifiers to select which thermogram values were most informative for segregating subjects. This represents an important initial step in determining what regions of the thermogram differ critically between cases and controls, and how these regions ‘interact’ or ‘contrast’ (i.e., as indicated by their coefficients). However, interpretation of the resulting coefficient profiles was challenging in certain cases, e.g. for the lasso penalized solutions. Hence, further work on decomposing the thermograms into salient and constituent peaks prior to applying classification approaches may improve model understanding while retaining full diagnostic utility. Such approaches are planned as future research.

There are several general comments concerning diagnostic classification models that need to be reiterated here. First, statistical significance does not imply clinical relevance or importance for predictive accuracy. With sufficient sample size, even minor differences that have low discriminatory power will appear statistically significant. Hence, when evaluating the utility of a diagnostic tool, predictive ability in addition to statistical significance must be considered. Second, classification accuracy must always be evaluated using an independent test set. Classification methods are particularly adept at finding solutions to discriminate between class labels, and evaluating the models based on data re-substitution will result in overly-optimistic (and often misleading) conclusions.

The results from this study show the critical importance in the development of diagnostic methods for the classification of clinical thermogram data. The growing number of studies applying DSC in multiple disease settings has served to illustrate the potential utility of DSC in characterizing clinical samples. Initial studies focused on straightforward approaches to correlate changes in thermogram features with clinical groups. Although consideration of certain thermogram features is useful in examining differences between groups, this study has shown that classification performance based on such measures (e.g., summary metrics) is limited. This study has evaluated a number of approaches for the diagnostic classification of DSC data but further development is needed in translating DSC towards clinical application. These approaches would also have to be amendable for clinical implementation in terms of generating a readily interpretable diagnostic result appropriate for the clinic setting. It is also critical to discover the association between biological disease processes and thermogram changes. Our prior studies have identified the “assignment” of peaks in the healthy thermogram through the study of individual purified plasma proteins. The situation in the disease state is much more complicated where modified thermal stabilities of major plasma proteins resulting from biomarker processes would result in complex thermogram changes. The accurate representation of these changes would serve to deconvolute the thermogram disease signature and provide an enhanced diagnostic approach focused on particular components or regions of the thermogram.

In conclusion, we have demonstrated that thermogram technology coupled with modern classification algorithms provides a powerful diagnostic approach for analysis of biological samples. Future work remains to develop an algorithm that is simultaneously interpretable while maintaining a high performance level. Uncovering the biological phenomena that drive the thermogram changes associated with a disease state will also lead to enhanced diagnostic approaches as well as make important biological discoveries which could improve our understanding of the underlying disease etiology.

### Example 2

Materials and Methods

Patient Population

De-identified plasma samples and patient data were obtained from the Lupus Family Registry and Repository (LFRR). Plasma samples for 300 patients meeting the revised criteria of the American College of Rheumatology for SLE and 300 healthy subjects matched demographically by sex, ethnicity and age (controls) were obtained from the LFRR. A patient is classified as having SLE if four of eleven ACR SLE criteria are present (Table 1). Plasma samples were received in frozen form on dry ice and were kept at −80° C. until thawed for DSC analysis. The LFRR data allow for the evaluation of any significant association of differences in thermograms with the ACR SLE criteria, as well as relevant demographic, serologic, and clinical data to evaluate influence of these covariates.

TABLE 1
SLE criteria evaluated in the study

| Criterion ¹ | Description | N (%) ² | |
|---|---|---|---|
| Serological ACR criteria | | | |
| 1. Immunological disorder | Positive Anti-dsDNA, Anti-Smith, or antiphospholipid | 246 | (82.0%) |
| | test (details below) | | |
| Anti-dsDNA | Autoantibodies to native double-stranded DNA. High | | |
| | specificity for SLE but low sensitivity. | | |
| Anti-Smith | Autoantibodies to Smith nuclear antigen. High specificity | | |
| | for SLE but low sensitivity. | | |
| Anti-cardiolipin IgG | Autoantibodies to cardiolipin, a mitochondrial membrane | | |
| | phospholipid, resulting in thrombosis. | | |
| Lupus anticoagulant | Autoantibodies that bind cell membrane phospholipids | | |
| | and proteins. Name derived from in vitro anticoagulant | | |
| | properties but in vivo interaction with platelet membrane | | |
| | phospholipids results in platelet aggregation and | | |
| | prothrombotic effects. | | |
| False +VDRL | A false positive venereal disease research laboratory | | |
| | (VDRL) test for at least 6 months confirmed by a | | |
| | Treponema pallidum immobilization or fluorescent | | |
| | treponemal antibody absorption test | | |
| 2. ANA titer | Autoantibodies to nuclear as well as cytoplasmic cell | 300 | (100%) |
| | components. High sensitivity for autoimmune diagnosis | | |
| | but low diagnostic specificity for SLE. | | |
| 3. Renal disorder | Either proteinuria or presence of cellular casts (details | 113 | (37.7%) |
| | below) | | |
| Proteinuria | Abnormal levels of protein in the urine indicating kidney | | |
| | dysfunction | | |
| Cellular casts | Cylindrical structures typically of red or white blood cells | | |
| | produced by the kidney and excreted into the urine. | | |
| | Indicates glomerular damage, inflammation or infection. | | |
| 4. Hematologic disorder | Presence of one of hemolytic anemia, leukopenia, | 201 | (67.0%) |
| | lyphopenia, or thrombocytopenia (details below) | | |
| Hemolytic anemia | Decreased red blood cell count as a result of autoantibody- | | |
| | mediated destruction. Common in about half of SLE | | |
| | patients. | | |
| Leukopenia | Decreased white blood cell count as a result of | | |
| | autoantibody-mediated destruction and indicative of an | | |
| | increased risk of infection. | | |
| Lyphopenia | Aka Lymphopenia, decreased levels of lymphocytes in the | | |
| | blood. Present in about 75% of SLE patients [1]. | | |
| Thrombocytopenia | Decreased platelet counts resulting from immune- | | |
| | mediated destruction or drug-impaired production. Mild | | |
| | thrombocytopenia observed in a quarter to a half of SLE | | |
| | patients. | | |
| Clinical ACR criteria | | | |
| 5. Malar rash | Rash localized on the nose and cheekbone (butterfly rash) | 133 | (43.3%) |
| | seen in about half of SLE patients. | | |
| 6. Discoid rash | Raised scaly rash on the head, arms, chest or back | 55 | (18.3%) |
| | observed in about a quarter of SLE patients. | | |
| 7. Photosensitivity | Skin rash as a result of unusual reaction to sunlight. | 141 | (47.0%) |
| 8. Oral ulcers | Mouth ulceration that is usually painless. | 91 | (30.3%) |
| 9. Arthritis | Tenderness and swelling of peripheral joints, usually hand | 250 | (83.3%) |
| | and wrist. | | |
| 10. Serositis | Inflammation of serous tissues, typically the lungs | 121 | (40.3%) |
| | (pleuritis) and heart (pericarditis). | | |
| 11. Neurologic disorder | Neurological symptoms including headaches, seizures and | 36 | (12.0%) |
| | psychosis resulting from damage to the central or | | |
| | peripheral nervous systems. | | |

¹ ACR criteria were scored on an integer scale from 0 to 3 based on increasing level of evidence that the clinical symptom is present. Subscales (e.g., 2A, 2B, 2C for Arthritis and Renal Disorder and 3A for Hematological and Immunological Disorders) are present for certain criteria. For each criteria, the variable corresponding to the highest value from medical records, patient interview, or other interview was used. A positive diagnosis of lupus is based on the presence of four of the eleven criteria. For details see Rassmussen et al. [2] and the references therein.
² Number and percent having convincing evidence (ACR criteria integer score = 3) that the criteria is met

DSC Sample Preparation

Samples were prepared according to our previously published procedure. Briefly, plasma samples (100 μL) were dialyzed against a standard phosphate buffer (1.7 mM KH₂PO₄, 8.3 mM K₂HPO₄, 150 mM NaCl, 15 mM sodium citrate, pH 7.5) for 24 hours at 4° C. in order to achieve normalization of buffer conditions for all samples. Samples were recovered from dialysis and filtered to remove particulates. The final dialysis buffer was also filtered and used for all sample dilutions and as a reference solution for DSC studies.

Collection of DSC thermograms

DSC data were collected according to our previously published procedure. Data were collected using an automated MicroCal VP-Capillary DSC instrument (MicroCal, LLC, Northampton, Mass., now a division of Malvern Instruments Inc.). Electrical calibration of the differential power signal and temperature calibration using hydrocarbon temperature standards were performed as part of the manufacturer annual instrument maintenance. Interim instrument performance was assessed using biological standards lysozyme and RNaseA. Dialyzed plasma samples were diluted 25-fold to obtain a suitable protein concentration for DSC analysis. Samples and dialysate were loaded into the instrument autosampler and thermostated at 5° C. until analysis. Thermograms were recorded from 20° C. to 110° C. at a scan rate of 1° C./min with a pre-scan thermostat of 15 minutes, mid feedback mode and a filtering period of 2 seconds. Duplicate thermograms were obtained for each plasma sample. DSC data were analyzed using Origin 7 (OriginLab Corporation, Northampton, Mass.). Raw data were corrected for the instrumental baseline by subtraction of a suitable buffer scan. Thermograms were normalized for total protein concentration and corrected for non-zero baselines by application of a linear baseline fit. Final thermograms were plotted as excess specific heat capacity (cal/° C.g) versus temperature (° C.).

Statistical Analysis of Thermograms

Thermograms were first visualized for differences between SLE patients and controls by plotting the mean±the 5^{th} and 95^{th} percentiles for each group at each temperature. To facilitate interpretation of the thermograms, several summary statistics including shape and feature metrics of the thermograms were calculated. These included principal components (PCs) of the thermograms, total area under the thermogram (range 45-90° C.), thermogram peak width at half height, maximum peak height, temperature of the peak maximum (Tₘₐₓ), maximum excess specific heat capacity (C_{P}^{ex}) of the first peak (Peak 1 max C_{P}^{ex}), maximum C_{P}^{ex} of the second peak (Peak 2 max C_{P}^{ex}), the ratio of (Peak 1 max C_{P}^{ex})/(Peak 2 max C_{P}^{ex}), and the first moment temperature T_{FM}. The T_{FM} was calculated as follows

$${T}_{FM} = \frac{{\int }_{45}^{90} ( {TC}_{p}^{ex} ) dT}{{\int }_{45}^{90} {C}_{p}^{ex} dT} .$$

Intuitively, the T_{FM} corresponds to a central mass point when considering the thermogram as a density curve.

Thermograms were subsequently stratified by important demographic, laboratory, and comorbidity data to determine whether these covariates influenced differences between SLE patients and controls. Differences between groups were tested for statistical significance by two-way ANOVA with interaction using the thermogram first PC as the response variable. The interaction term was used to determine whether a covariate influenced any differences in thermograms between SLE patients and controls. Differences in the first PC were also tested for according to each of the SLE diagnostic criteria (Table 1), serology (Anti dsDNA titer, Anti Ro, Anti La, Anti Smith, ANA titer, and Anti-cardiolipin immunoglobulin G and M), number of ACR criteria and type of SLE onset, patient medications (Prednisone and Hydroxychloroquine), and additional labs (complement C3/C4, hemoglobin, white blood cell/lymphocyte/platelet count, erythrocyte sedimentation rate, globulin, proteinuria, albumin, creatinine, and creatinine clearance) among SLE patients only, to evaluate whether thermogram measures correlated with a certain aspect of SLE or other serological/laboratory data. P-values were adjusted for multiple comparisons based on the false-discovery rate (FDR) correction.

Next, an evaluation as to whether the observed differences in thermograms between SLE patients and controls had any diagnostic utility was conducted. A modified version of Fisher's linear discriminant analysis (MLDA) was used to classify subjects as SLE versus control using the information from the thermograms. The MLDA classifier was designed to handle situations where the number of variables (here, excess specific heat capacity at each temperature) potentially exceeds the number of subjects. Determination of SLE was based on the posterior probability of SLE given the thermogram data, as outputted from the MLDA algorithm. Classification based on thermograms alone used a threshold probability of 0.5, while coupling thermogram information together with SLE serological markers used a more stringent threshold of 0.9 (since the goal was to catch cases not detected by the immunological markers).

Results

Comparison of Thermograms Between SLE Patients and Controls

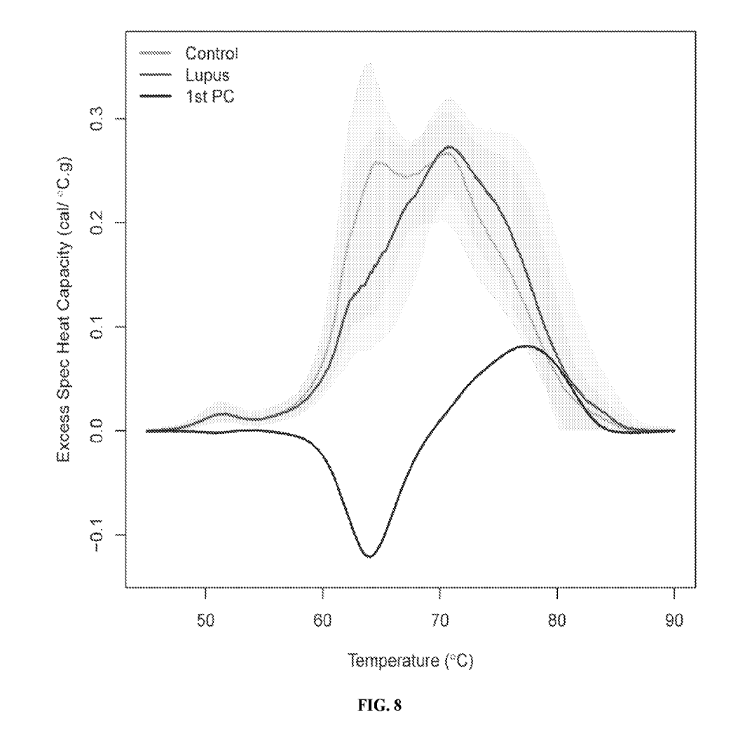

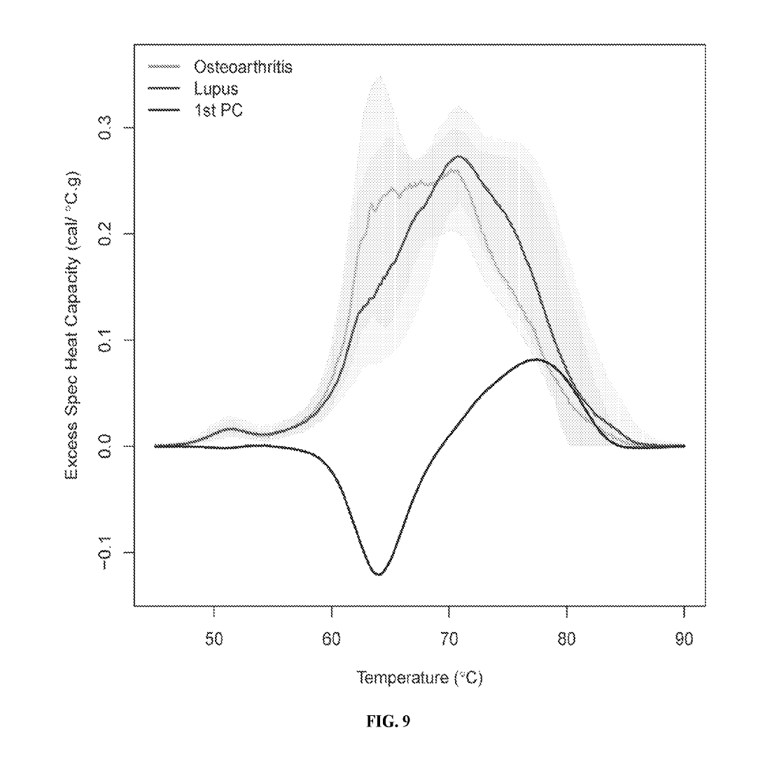

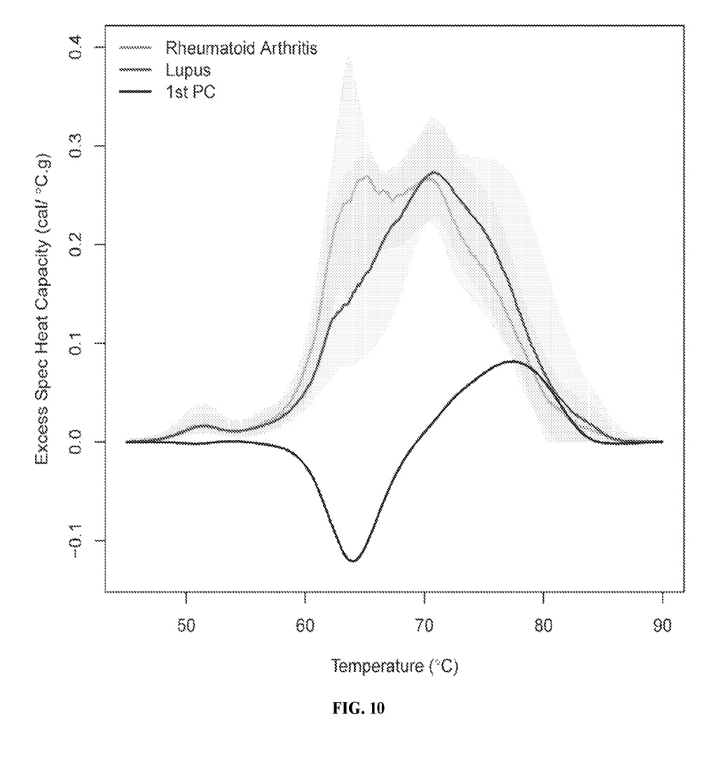

A graphical display of the average thermograms separately for SLE patients and controls revealed significant differences between the two sets of subjects (FIG. 8). In particular, the average thermogram for SLE patients has a markedly reduced initial peak corresponding to −65° C. and the second peak around 70-75° C. is shifted to the right relative to the control subjects. To examine the ability of thermograms to distinguish SLE from other autoimmune diseases thermograms of SLE patients were compared to controls with autoimmune comorbidities. The thermograms for SLE patients also differed in a similar fashion from controls with osteoarthritis (n=31 subjects, FIG. 9) and rheumatoid arthritis (n=16 subjects, FIG. 10). An overlay of the first principal component (PC) indicates that the loadings for the 1^{st} PC correspond with the shift in the two major peaks seen in the two sets of thermograms. A scree plot for the PCs indicated that six PCs were sufficient to characterize the variability in the thermograms (98.2% of total variability explained, see FIG. 11). A multivariate test of differences between SLE and control subjects based on the first six PCs was highly significant (p≤10⁻¹⁵), as was the test based on only the first PC (p<10⁻¹⁵).

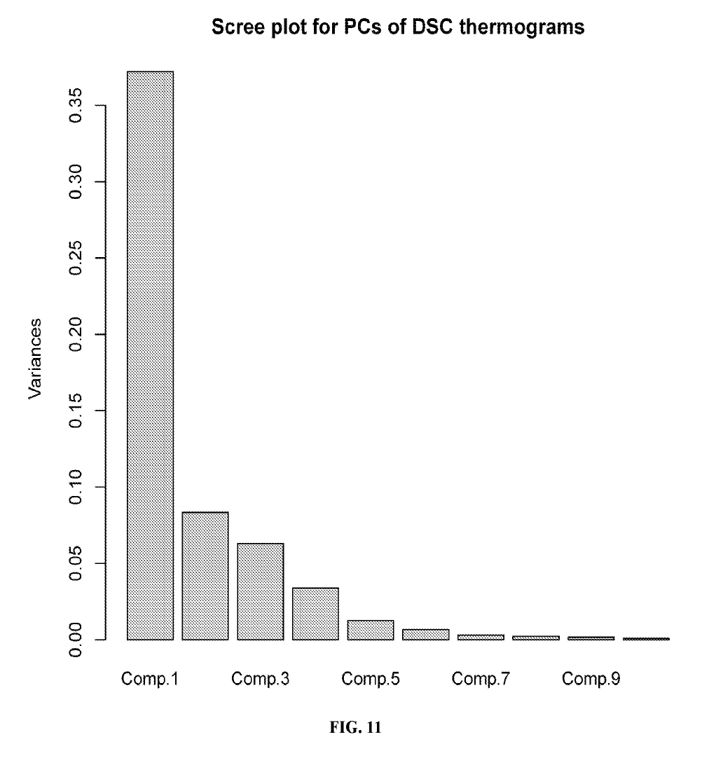

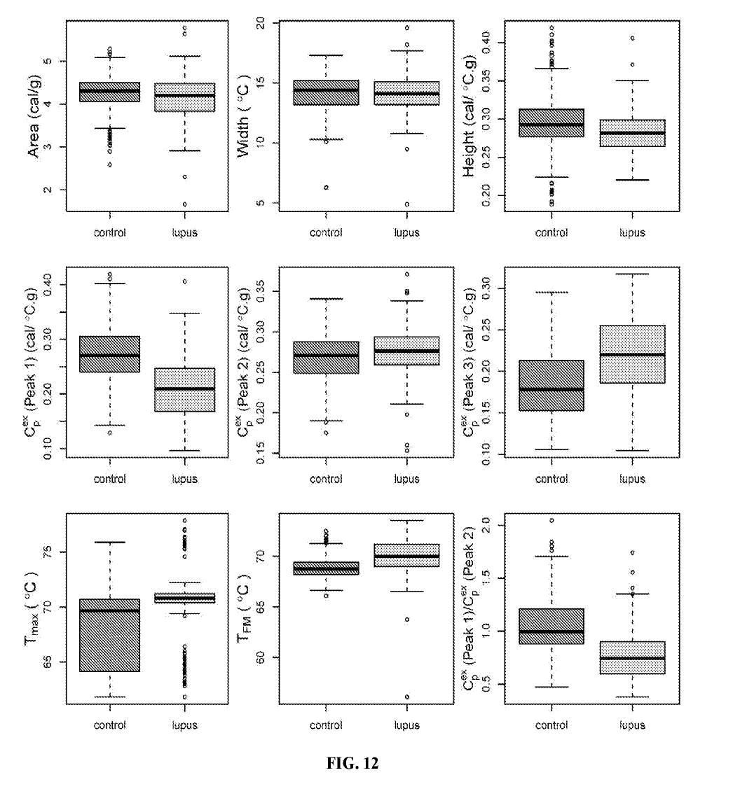

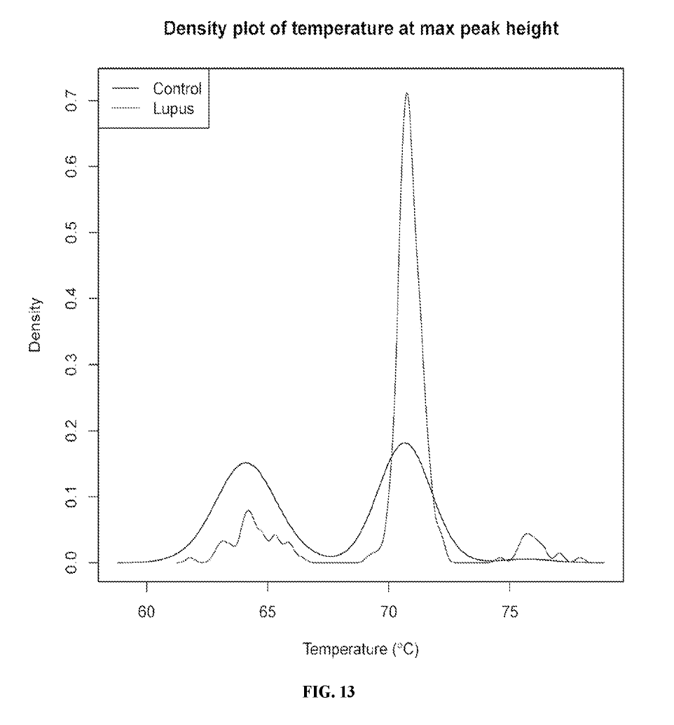

Thermogram summary statistics (as described in the Methods) were calculated and compared between SLE patients and controls (FIG. 12). A density plot of Tₘₐₓ revealed that there were roughly three prominent peaks among the subjects at 62-67° C., 69-73° C., and 75-80° C. (FIG. 13). Highly significant differences (p<0.001, based on the t-test) were present for maximum peak height, Tₘₐₓ, Peak 1 max C_{P}^{ex}, Peak 2 max C_{P}^{ex}, the ratio of (Peak 1 max C_{P}^{ex})/(Peak 2 max C_{P}^{ex}), and T_{FM}. The thermogram peak width at half height was not significantly different between the two groups (p=0.68), while the total area under the thermogram was slightly higher for controls (p=0.035). Distinct subpopulations of SLE patients based on differences in Tₘₐₓ are observed resulting from variability in the distribution of the thermogram profile. This observation might be related to the clinical status of the patients (for example, active flare versus no flare; with kidney disease versus without kidney disease) and may represent an important application of thermograms for clinical monitoring of these patients.

Influence of Covariates on Thermogram Differences

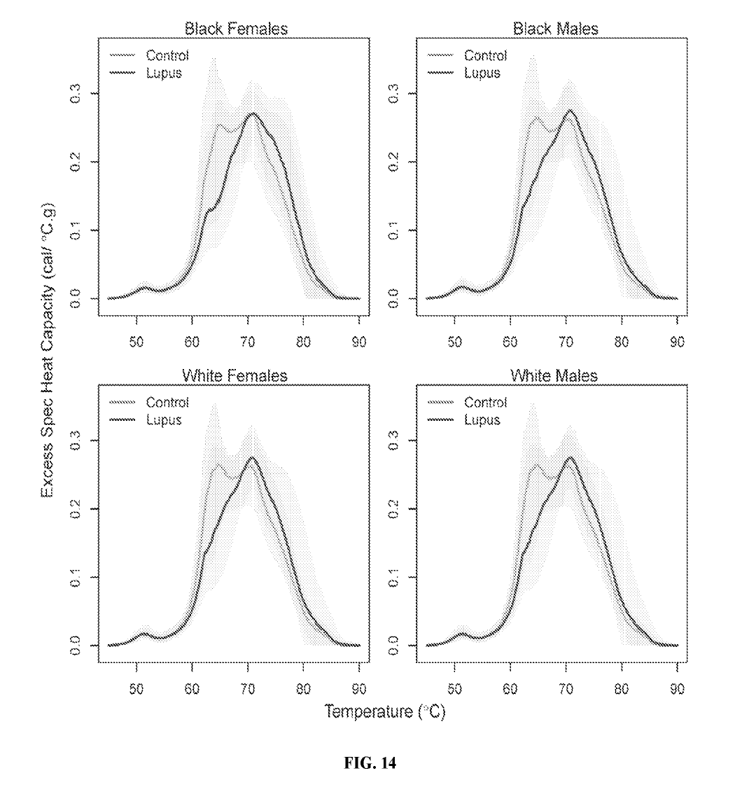

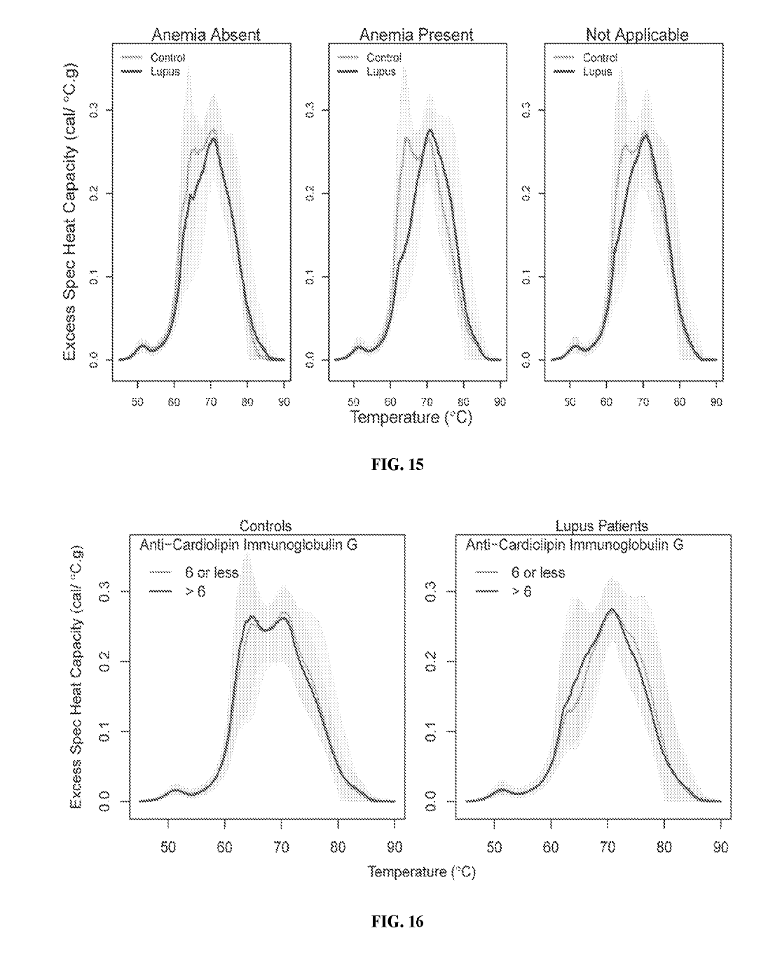

Differences between SLE patients and controls along important demographic factors and comorbidities are detailed in Table 2. Demographic factors were similar between the two groups, while expectedly conditions associated with SLE differed. These covariates were subsequently evaluated to determine whether they were effect modifiers for the differences in thermograms between SLE patients and controls, by investigating the significance of the interaction term between the covariate and case/control status in a linear model with the first PC of the thermograms as the response. Of the 22 variables listed in Table 2, only sex and anemia were found to have a statistically significant interaction with case/control status after adjusting for multiple comparisons (FDR adjusted p<0.001 and 0.02, respectively; see Table 3). Ethnicity had a significant unadjusted p-value (p=0.04), but was not significant after accounting for multiple comparisons. The interaction can be demonstrated visually by viewing average thermogram profiles for SLE and control subjects stratified by sex and ethnicity (FIG. 14) and anemia (FIG. 15). Separation between SLE and control subjects is more evident for females, black ethnicity, and subjects with anemia. In a similar fashion, we investigated whether variations in thermograms among SLE patients were associated with any of the SLE diagnostic criteria and additional laboratory data (Table 4). The most significant result was for anti-cardiolipin immunoglobulin G (IgG, FDR adjusted p=0.10). Patients with higher IgG values had thermograms shifted to the left, which was true for both SLE patients and controls (FIG. 16).

TABLE 2
Demographics and Comorbidities/Other Conditions by Case Status

| | Control N (%) | | Lupus N (%) | |
|---|---|---|---|---|
| Demographics | | | | |
| Gender | | | | |
| Female | 225 | (75) | 226 | (75.3) |
| Male | 75 | (25) | 74 | (24.7) |
| Ethnicity | | | | |
| Black | 157 | (52.3) | 159 | (53) |
| White | 139 | (46.3) | 141 | (47) |
| Other | 4 | (1.3) | 0 | (0) |
| Year of birth | | | | |
| (1924, 1944] | 70 | (23.6) | 87 | (29.5) |
| (1944, 1955] | 68 | (23) | 78 | (26.4) |
| (1955, 1971] | 80 | (27) | 64 | (21.7) |
| (1971, 1993] | 78 | (26.4) | 66 | (22.4) |
| BMI | | | | |
| [6, 16] | 1 | (0.4) | 0 | (0) |
| (16, 18.5] | 7 | (2.8) | 13 | (5.4) |
| (18.5, 25] | 86 | (34.8) | 89 | (36.9) |
| (25, 30] | 76 | (30.8) | 61 | (25.3) |
| (30, 71] | 77 | (31.2) | 78 | (32.4) |
| Smoking now | | | | |
| No | 78 | (32) | 77 | (34.1) |
| Yes | 34 | (13.9) | 41 | (18.1) |
| Not applicable | 132 | (54.1) | 108 | (47.8) |
| Number of years smoking | | | | |
| [0, 10] | 24 | (10.1) | 16 | (7.4) |
| (10, 20] | 25 | (10.5) | 25 | (11.6) |
| (20, 30] | 21 | (8.8) | 24 | (11.1) |
| (30, 40] | 13 | (5.5) | 24 | (11.1) |
| (40, 50] | 15 | (6.3) | 17 | (7.9) |
| (50, 60] | 140 | (58.8) | 110 | (50.9) |
| Comorbidities/Other Conditions | | | | |
| High blood pressure | | | | |
| No | 163 | (66) | 121 | (43.2) |
| Yes | 84 | (34) | 159 | (56.8) |
| Arthritis (current or past) | | | | |
| No | 162 | (66.1) | 70 | (25.1) |
| Yes | 83 | (33.9) | 209 | (74.9) |
| Osteoarthritis | | | | |
| No | 209 | (87.1) | 221 | (82.8) |
| Yes | 31 | (12.9) | 46 | (17.2) |
| Rheumatoid arthritis | | | | |
| No | 226 | (93.4) | 156 | (58.2) |
| Yes | 16 | (6.6) | 112 | (41.8) |
| Low blood count | | | | |
| No | 169 | (67.6) | 66 | (24) |
| Yes | 81 | (32.4) | 209 | (76) |
| Anemia | | | | |
| No | 28 | (11.5) | 58 | (22.4) |
| Yes | 67 | (27.5) | 156 | (60.2) |
| Not applicable | 149 | (61.1) | 45 | (17.4) |
| Hemolytic anemia | | | | |
| No | 81 | (35.1) | 165 | (73.7) |
| Yes | 1 | (0.4) | 14 | (6.2) |
| Not applicable | 149 | (64.5) | 45 | (20.1) |
| Low white blood cell count | | | | |
| No | 63 | (26.9) | 63 | (25.7) |
| Not applicable | 149 | (63.7) | 44 | (18) |
| Yes | 22 | (9.4) | 138 | (56.3) |
| Low number of platelets | | | | |
| No | 71 | (30.6) | 80 | (33.8) |
| Yes | 12 | (5.2) | 113 | (47.7) |
| Not applicable | 149 | (64.2) | 44 | (18.6) |
| Mononucleosis | | | | |
| No | 231 | (93.9) | 249 | (90.9) |
| Yes | 15 | (6.1) | 25 | (9.1) |
| Psoriasis | | | | |
| No | 230 | (95.4) | 235 | (86.4) |
| Yes | 11 | (4.6) | 37 | (13.6) |
| Scleroderma | | | | |
| No | 242 | (99.6) | 259 | (95.9) |
| Yes | 1 | (0.4) | 11 | (4.1) |
| Recurrent chest pain | | | | |
| No | 209 | (87.1) | 129 | (55.6) |
| Yes | 31 | (12.9) | 103 | (44.4) |
| Heart attack | | | | |
| No | 235 | (96.7) | 249 | (90.5) |
| Yes | 8 | (3.3) | 26 | (9.5) |
| Cancer | | | | |
| No | 232 | (96.3) | 211 | (88.3) |
| Yes | 9 | (3.7) | 28 | (11.7) |
| Diabetes | | | | |
| No | 214 | (87.3) | 244 | (88.4) |
| Yes | 31 | (12.7) | 32 | (11.6) |

TABLE 3
P-values for interaction between covariate and case/control
status in a statistical model with the first PC of
the thermograms as the response variable

| | | FDR¹ |
|---|---|---|
| | Unadjusted | adjusted |
| | p-value | p-value |
| Demographics | | |
| Gender | <0.001 | <0.001 |
| Ethnicity | 0.04 | 0.31 |
| Year of birth | 0.96 | 0.97 |
| BMI | 0.48 | 0.82 |
| Smoking now | 0.87 | 0.97 |
| Number of years smoking | 8.59 | 0.84 |
| Comorbidities/Other Conditions | | |
| High blood pressure | 0.08 | 0.40 |
| Arthritis (current or past) | 0.51 | 0.82 |
| Osteoarthritis | 0.68 | 0.91 |
| Rheumatoid arthritis | 0.29 | 0.70 |
| Low blood count | 0.23 | 0.62 |
| Anemia | 0.002 | 0.02 |
| Hemolytic anemia | 0.06 | 0.36 |
| Low white blood cell count | 0.19 | 0.62 |
| Low number of platelets | 0.16 | 0.62 |
| Mononucleosis | 0.46 | 0.82 |
| Psoriasis | 0.95 | 0.97 |
| Scleroderma | 0.39 | 0.78 |
| Recurrent chest pain | 0.56 | 0.83 |
| Heart attack | 0.92 | 0.97 |
| Cancer | 0.87 | 0.97 |
| Diabetes | 0.21 | 0.62 |

¹FDR = False Discovery Rate

TABLE 4
P-values for association between the first PC of
the thermograms and ACR diagnostic criteria listed
in Supplementary Table 1 among SLE patients

| | | FDR¹ |
|---|---|---|
| | Unadjusted | adjusted |
| | p-value | p-value |
| Serological ACR criteria ² | | |
| 1. Immunological disorder (highest)³ | 0.42 | 0.70 |
| Anti-dsDNA (OMRF serology) ⁴ | 0.19 | 0.60 |
| Anti-Smith (OMRF serology) | 0.37 | 0.68 |
| Antiphospholipid Ab ⁵ (highest) | 0.016 | 0.28 |
| Anti-cardiolipin IgG (OMRF serology) | 0.29 | 0.67 |
| Lupus anticoagulant (med record) ⁶ | 0.10 | 0.53 |
| False +VDRL (med record) | 0.43 | 0.71 |
| 2. ANA titer (highest) ⁷ | — | — |
| 3. Renal disorder (highest) | 0.60 | 0.80 |
| Proteinuria (med record) | 0.21 | 0.60 |
| Cellular casts (med record) | 0.60 | 0.80 |
| 4. Hematologic disorder (highest) | 0.79 | 0.91 |
| Hemolytic anemia (med record) | 0.10 | 0.53 |
| Leukopenia (med record) | 0.89 | 0.94 |
| Lyphopenia (med record) | 0.16 | 0.60 |
| Thrombocytopenia (med record) | 0.71 | 0.86 |
| Clinical ACR criteria | | |
| 5. Malar rash (highest) | 0.08 | 0.50 |
| 6. Discoid rash (highest) | 0.18 | 0.60 |
| 7. Photos ensitivity (highest) | 0.98 | 1.00 |
| 8. Oral ulcers (highest) | 0.06 | 0.42 |
| 9. Arthritis (highest) | 0.30 | 0.67 |
| 10. Serositis (highest) | 0.22 | 0.60 |
| Pericarditis (med record) | 0.81 | 0.92 |
| Pleuritis (med record) | 0.03 | 0.35 |
| 11. Neurologic disorder (highest) | 0.05 | 0.39 |
| Seizures (med record) | 0.14 | 0.56 |
| Psychosis (med record) | 0.13 | 0.56 |
| Other SLE related criteria | | |
| Number ACR criteria (med records and OMRF | 0.13 | 0.56 |
| serology) | | |
| Type SLE onset (acute, insidious, or | 0.28 | 0.67 |
| indeterminate) | | |
| Additional autoimmune illness (y/n) | 0.64 | 0.80 |
| OMRF antibody laboratory testing | | |
| Anti-dsDNA titer (highest test value documented) | 0.31 | 0.67 |
| Anti-Smith (positive or negative) | 0.33 | 0.67 |
| Anti-Ro (positive or negative) | 0.55 | 0.79 |
| Anti-La (positive or negative) | 0.98 | 1.00 |
| ANA titer (highest test value documented) | 0.46 | 0.73 |
| Anti-cardiolipin immunoglobulin G (highest test | 0.002 | 0.10 |
| value documented) | | |
| Anti-cardiolipin immunoglobulin M (highest test | 0.007 | 0.19 |
| value documented) | | |
| Additional laboratory testing | | |
| Complement C3 (lowest test value documented) | 0.63 | 0.80 |
| Complement C4 (lowest test value documented) | 0.52 | 0.77 |
| Hemoglobin (lowest test value documented) | 0.62 | 0.80 |
| White blood cell (lowest test value documented) | 0.38 | 0.68 |
| Lymphocyte count (lowest test value documented) | 0.38 | 0.68 |
| Platelet count (lowest test value documented) | 0.33 | 0.67 |
| Erythrocyte sedimentation rate (highest test | 0.87 | 0.94 |
| value documented) | | |
| Globulin (highest test value documented) | 0.64 | 0.80 |
| Proteinuria measured in mg/24 hours (highest | 0.21 | 0.60 |
| test value documented) | | |
| Albumin (lowest test value documented) | 0.52 | 0.77 |
| Creatinine (highest test value documented) | 0.88 | 0.94 |
| Creatinine clearance (lowest test value | 0.74 | 0.88 |
| documented) | | |
| Patient medications | | |
| Prednisone (Deltasone, Meticorten) | 0.40 | 0.69 |
| Hydroxychloroquine (Plaquenil) | 0.04 | 0.39 |

¹FDR = False Discovery Rate
² Tests of association here are based on an ACR criteria evidence score (0-3). These values may differ from the ‘OMRF antibody laboratory testing’ section where in that section they are based on the reported laboratory result (either a titer value or positive/negative determination).
³Highest = Maximum score between the medical records, subject interview, or other medically convincing source
⁴ OMRF serology = Based on OMRF serological testing
⁵ Summary of antiphospholipid antibody tests showing highest score of false +VDRL, lupus anticoagulant, and anticardiolipin antibody documented via subject interview, physician interview, medical records, or other medically convincing source
⁶ Med record = Based on information in the medical record
⁷ No test could be done for ACR criteria of ANA titer since all lupus patients were found to have convincing evidence of this. In contrast a test for association with actual ANA titer values (under ‘OMRF antibody laboratory testing’) is possible.

Classification of SLE Patients Versus Controls

To determine whether the observed differences in thermograms between SLE patients and controls had any diagnostic utility, the MLDA program was used to classify subjects as SLE versus control based on the information from the thermograms. Three different diagnostic models were compared: a) a model based on DSC thermograms only (DSC), b) a model based on antibody tests only (Ab), and c) a model based on coupling the antibody test with thermograms (DSC+Ab). For purposes of establishing a biomarker based comparator we selected one which had optimal performance (accuracy) in our data. That is, we have classified a subject as SLE positive if any of the Anti dsDNA titer, Anti Ro, Anti La, Anti Smith, and ANA titer tests were positive. Specifically, a titer of 1:30 or higher was considered positive for the Anti dsDNA test, while a value of 1:360 or higher was considered positive for the ANA titer. The high value for the ANA titer was used to achieve optimal overall accuracy (otherwise, sensitivity will be 100% but specificity will be much lower). All other lab values were simply reported as positive or negative in the LFRR database.

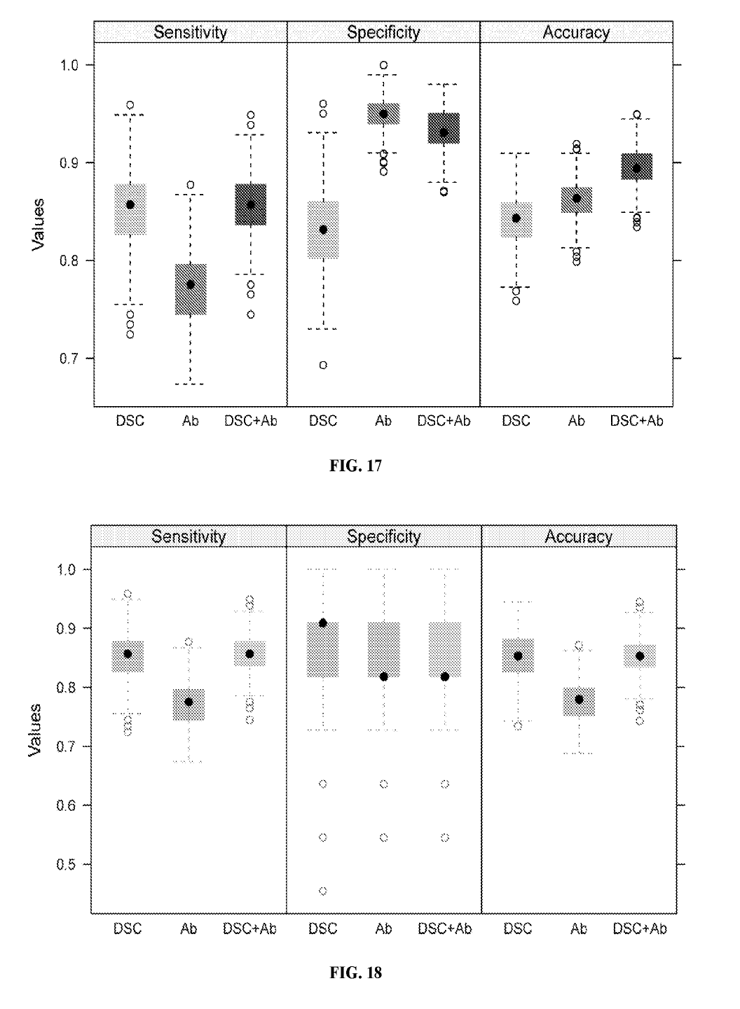

All of these tests are highly specific for SLE but have low sensitivity, so combining them in this fashion produced the optimal test based on antibodies alone. Data were randomly split 1000 times into training (two thirds) and test (one third) data sets, and sensitivity, specificity, and overall accuracy were calculated for each model for each split. Median sensitivity, specificity, and overall accuracy based on classification using plasma thermograms was 86%, 83%, and 84% compared to 78%, 95%, and 86% based on the combined serological markers (FIG. 17). To combine the models and improve the sensitivity of the antibody based test while maintaining the same specificity, a subject was classified as SLE positive if either the antibody test was positive or the predicted probability of SLE based on the thermogram passed a high threshold (probability >0.9). Results indicate that the sensitivity (86%) and overall accuracy (89%) were improved relative to the antibody only test, with only a small drop in specificity (93%) (FIG. 17).

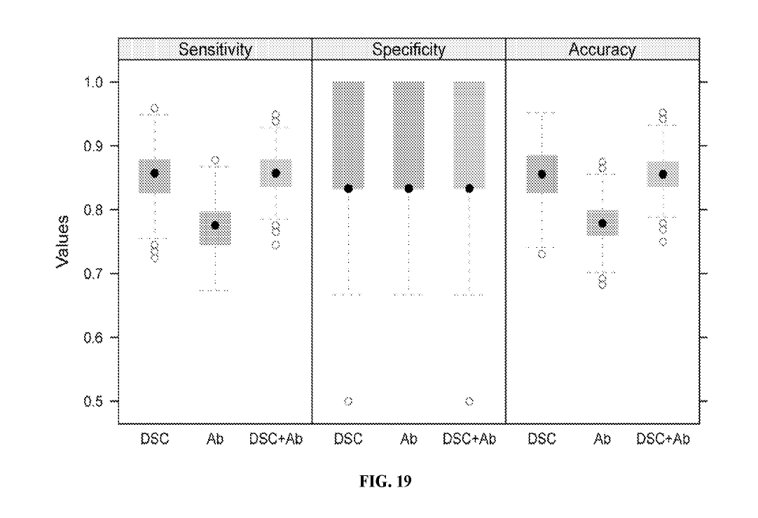

The classification performance of the instant DSC-based and combined DSC+Ab model was also investigated on specific subsets of patients. FIGS. 18 and 19 show that comparable results were obtained when classifying SLE patients versus controls with a comorbidity of either osteoarthritis or rheumatoid arthritis, with median overall accuracies based on the DSC profiles of 85% and 86%, respectively. Median accuracy and inter-quartile range (IQR) for the DSC and antibody based models stratified by gender and ethnicity are given in Table 5. DSC accuracy was lower among white males compared to the other three sex/ethnic groups. However, in all cases combining DSC and antibody information improved the overall accuracy relative to the Ab only models by increasing the overall sensitivity. This increase was most pronounced in white females, where sensitivity improved from 68% in the Ab only model to 80% in the DSC+Ab model and overall accuracy improved from 79% to 84%. In most cases (the exception being white males), the specificity of the combined DSC+Ab model was only slightly impacted (1% decrease or less) relative to the Ab only model.

TABLE 5
Accuracy of DSC, antibody only, and combined antibody +
DSC classifiers in patient subsets according to race and
gender. Entries in each cell are median and inter-quartile range (IQR,
25^{th} percentile and 75^{th} percentile).

| | Black Females | Black Males | White Females | White Males |
|---|---|---|---|---|
| | DSC | | | |
| Sensitivity | 0.89 | 0.86 | 0.86 | 0.75 |
| | (0.86, 0.92) | (0.75, 0.91) | (0.82, 0.90) | (0.68, 0.83) |
| Specificity | 0.84 | 0.88 | 0.87 | 0.73 |
| | (0.80, 0.87) | (0.80, 1.00) | (0.83, 0.91) | (0.65, 0.80) |
| Accuracy | 0.86 | 0.86 | 0.87 | 0.74 |
| | (0.84, 0.89) | (0.80, 0.91) | (0.84, 0.89) | (0.69, 0.79) |
| | Antibody Only | | | |
| Sensitivity | 0.87 | 0.78 | 0.68 | 0.69 |
| | (0.84, 0.90) | (0.67, 0.86) | (0.63, 0.72) | (0.62, 0.75) |
| Specificity | 0.97 | 1.00 | 0.9 | 1.00 |
| | (0.95, 0.98) | (0.90, 1.00) | (0.87, 0.93) | (0.95, 1.00) |
| Accuracy | 0.92 | 0.87 | 0.79 | 0.84 |
| | (0.90, 0.94) | (0.81,0.91) | (0.76, 0.82) | (0.80, 0.87) |
| | Antibody + DSC | | | |
| Sensitivity | 0.93 | 0.85 | 0.80 | 0.78 |
| | (0.91, 0.95) | (0.75, 0.90) | (0.75, 0.85) | (0.72, 0.83) |
| Specificity | 0.96 | 1.00 | 0.89 | 0.94 |
| | (0.93, 0.98) | (0.90, 1.00) | (0.86, 0.92) | (0.92, 1.00) |
| Accuracy | 0.94 | 0.89 | 0.84 | 0.86 |
| | (0.93, 0.96) | (0.85, 0.94) | (0.82, 0.87) | (0.83, 0.90) |

Discussion

DSC analysis of biofluid samples is an emerging area of proteomics research with demonstration of preliminary utility for the discrimination of disease subjects from controls in multiple disease types. To further investigate these observations, we embarked upon a much larger study to confirm the utility of DSC for discrimination of SLE from controls and to evaluate additional demographic and clinical factors of interest. Further, this study is the first to demonstrate how thermograms can be used to improve upon an existing serological based classification, here by increasing both sensitivity and overall accuracy for SLE patients versus controls. This gives a template for developing thermograms as a potential complementary diagnostic tool.

Application of the MLDA approach to thermogram data determined median diagnostic sensitivity, specificity and overall accuracy of 86%, 83% and 84%, respectively, for the classification of SLE patients versus healthy controls. These results compare well to the study by Fish et al. and Garbett and Brock where median overall accuracy ranged from 74-88%. Further, by including information from thermograms the median sensitivity of the defined antibody test for SLE was improved from 78% to 86% and the overall accuracy improved from 86% to 89%, while the specificity was minimally impacted (reduced from 905% to 93%). The classification cut-offs for the thermogram data were based on posterior probabilities as determined by the MLDA algorithm, and a noted limitation of the current approach is the difficulty in interpreting the resulting thermogram ‘signature’ for separating cases and controls (c.f. FIG. 7 in Garbett and Brock).

The ability of the thermograms to accurately classify SLE patients varied according to several demographic factors (sex, ethnicity) and other health conditions (anemia), with highest accuracy in females and black subjects. In our previous study of 100 healthy plasma samples we also found differences in thermograms according to sex and ethnicity, and these variations can form the basis for development of specific healthy control populations. However, in every case the overall accuracy of the thermogram based models was comparable to the optimal antibody based test and the combined antibody/thermogram models improved the sensitivity and overall accuracy of the antibody tests. No other statistically significant demographic/health-related factors were identified which impacted the thermogram differences between SLE patients and controls. That is, similar differences in thermograms were observed between SLE and control subjects with high blood pressure, arthritis, mononucleosis, recurrent chest pain, diabetes, and cancer (c.f Table 3). However, in some of these cases (e.g., cancer) the number of subjects with the condition is too small to make definitive conclusions. And while it was determined that thermogram profiles for SLE patients differed from controls with osteoarthritis and rheumatoid arthritis, the instant inventors were unable to investigate whether SLE patient profiles differed from other important connective tissue diseases such as primary Sjogren's syndrome (n=4 patients in our data). Lastly, patient medications (prednisone and hydroxychloroquine) did not influence thermogram changes resulting from SLE status, although this would require additional testing.

Use of plasma samples from the LFRR repository is extremely valuable in exploring the potential of DSC analysis for detection of SLE but has limitations which prevent the full diagnostic and prognostic utility of DSC profiling from being observed. First, a patient's current disease status (e.g., different organ involvement, disease remission vs. flare) and measures of disease activity (see, e.g., Romero-Diaz et al.) at the time of blood sample collection are not recorded in the database, and this can impact the thermogram. Temporal variation may be observed in thermograms which can potentially be correlated with changes in the physiological state of the disease. Evaluating how thermogram changes track with disease severity over time are important for determining the full clinical applicability of DSC profiling. Lastly, the determination of SLE in the LFRR database is based on the ACR criteria, and the revised SLICC classification criteria may have increased sensitivity for SLE. However, despite the limitations of the LFRR data, thermogram classification is comparable to antibody based testing and improves the overall accuracy when the two tests are combined.

The aforementioned host of SLE phenotypes, serology, and laboratory measurements were conducted to uncover potential biophysical underpinnings of thermograms differences between SLE patients and controls. The most prominent association was somewhat counter-intuitive, in that patients with lower anti-cardiolipin IgG and IgM levels had thermograms with a more prominent transition around 75-80° C., a region of the thermogram previously described as dominated by immunoglobulin transitions. The same observation was also noted among controls. None of the other candidates investigated (including C3/C4 complements, ANA titers, etc.) were significantly associated with thermogram shifts. However, a stronger separation between SLE patients and controls was observed among those with anemia. One potential explanation for this finding is the expected association between anemia and thrombocytopenia in SLE, where the latter is usually a marker of more severe disease. However, thrombocytopenia alone was not associated with thermogram shifts. In sum, this study was a valuable first step in exploring the mechanism of thermogram modulation in SLE.

One could envision several ways to apply DSC in a clinical setting. First, DSC could be used as another measure to confirm a case of SLE when antibody tests are all negative but other clinical symptoms are present. In this case using DSC with a high threshold probability is warranted to maintain high specificity for SLE. Alternatively, DSC could be considered as a single test alternative to the suite of antibody tests, based on the overall sensitivity and specificity of DSC alone for SLE. In this case, a lower threshold probability is needed for SLE to ensure a high enough sensitivity. Lastly, DSC could be applied primarily for detection within certain demographic groups where antibody-based tests are less effective (e.g., white females based on our results). There is also an unmet need for early SLE diagnosis, particularly for cases presenting with <4 ACR criteria but with major organ disease, as well as for SLE surveillance, particularly for early detection of changes in disease activity, organ involvement or therapeutic response.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

[1] A. Cooper, M. A. Nutley, A. Wadood, Differential scanning microcalorimetry, in: B. Z. Chowdhry, S. E. Harding (Eds.) Protein-Ligand Interactions: hydrodynamics and calorimetry: a practical approach, Oxford University Press, Oxford, U K, 2001, pp. 287-318.
[2] C. M. Johnson, Differential scanning calorimetry as a tool for protein folding and stability, Arch. Biochem. Biophys., 531 (2013) 100-109.
[3] N. C. Garbett, C. S. Mekmaysy, L. DeLeeuw, J. B. Chaires, Clinical application of plasma thermograms. Utility, practical approaches and considerations, Methods, 76 (2015) 41-50.
[4] N. C. Garbett, C. S. Mekmaysy, C. W. Helm, A. B. Jenson, J. B. Chaires, Differential scanning calorimetry of blood plasma for clinical diagnosis and monitoring, Exp. Mol. Pathol., 86 (2009) 186-191.
[5] N. C. Garbett, J. J. Miller, A. B. Jenson, J. B. Chaires, calorimetry outside the box: a new window into the plasma proteome, Biophys. J., 94 (2008) 1377-1383.
[6] N. C. Garbett, J. J. Miller, A. B. Jenson, J. B. Chaires, calorimetric Analysis of the Plasma Proteome, Semin. Nephrol., 27 (2007) 621-626.
[7] N. C. Garbett, J. J. Miller, A. B. Jenson, D. M. Miller, J. B. Chaires, Interrogation of The Plasma Proteome with Differential Scanning calorimetry, Clin. Chem., 53 (2007) 2012-2014.
[8] N. C. Garbett, J. J. Miller, A. B. Jenson, J. B. Chaires, Ligand Binding Alters the calorimetric Thermogram of Albumin, J. Clin. Ligand Assay, 29 (2006) 194-197.
[9] N. C. Garbett, M. L. Merchant, J. B. Chaires, J. B. Klein, calorimetric analysis of the plasma proteome: Identification of type 1 diabetes patients with early renal function decline, Biochim Biophys Acta, 1830 (2013) 4675-4680.
[10] N. C. Garbett, M. L. Merchant, C. W. Helm, A. B. Jenson, J. B. Klein, J. B. Chaires, Detection of Cervical Cancer Biomarker Patterns in Blood Plasma and Urine by Differential Scanning calorimetry and Mass Spectrometry, PLOS ONE, 9 (2014) e84710.
[11] A. A. Chagovetz, R. L. Jensen, L. Recht, M. Glantz, A. M. Chagovetz, Preliminary use of differential scanning calorimetry of cerebrospinal fluid for the diagnosis of glioblastoma multiforme, J Neurooncol, 105 (2011) 499-506.
[12] A. A. Chagovetz, C. Quinn, N. Damarse, L. D. Hansen, A. M. Chagovetz, R. L. Jensen, Differential scanning calorimetry of gliomas: a new tool in brain cancer diagnostics?, Neurosurgery, 73 (2013) 289-295.
[13] T. Fekecs, I. Zapf, A. Ferencz, D. Lörinczy, Differential scanning calorimetry (DSC) analysis of human plasma in melanoma patients with or without regional lymph node metastases, J. Therm. Anal. calorim., 108 (2012) 149-152.
[14] A. Ferencz, T. Fekecs, D. Lörinczy, Differential Scanning calorimetry, as a New Method to Monitor Human Plasma in Melanoma Patients with Regional Lymph Node or Distal Metastases, in: Y. Xi (Ed.) Skin Cancer Overview, InTech, 2011, pp. 141-151.
[15] D. J. Fish, G. P. Brewood, J. S. Kim, N. C. Garbett, J. B. Chaires, A. S. Benight, Statistical analysis of plasma thermograms measured by differential scanning calorimetry, Biophys. Chem., 152 (2010) 184-190.
[16] S. Krumova, B. Rukova, S. Todinova, L. Gartcheva, V. Milanova, D. Toncheva, S. G. Taneva, calorimetric monitoring of the serum proteome in schizophrenia patients, Thermochim. Acta, 572 (2013) 59-64.
[17] M. Mehdi, T. Fekecs, I. Zapf, A. Ferencz, D. Lörinczy, Differential scanning calorimetry (DSC) analysis of human plasma in different psoriasis stages, J. Therm. Anal. calorim., 111 (2013) 1801-1804.
[18] A. Michnik, Blood plasma, serum and serum proteins microcalorimetric studies aimed at diagnosis support, in: D. Lörinczy (Ed.) Thermal Analysis in Medical Application, Akad\tilde{e}miai Kiadõ, Budapest, Hungary, 2011, pp. 171-190.
[19] A. Michnik, Z. Drzazga, K. Michalik, A. Barczyk, I. Santura, E. Sozaliska, W. Pierzchala, Differential scanning calorimetry study of blood serum in chronic obstructive pulmonary disease, J. Therm. Anal. calorim., 102 (2010) 57-60.
[20] A. Michnik, Z. Drzazga, S. Poprzecki, M. Czuba, K. Kempa, E. Sadowska-Krepa, DSC serum profiles of sportsmen, J. Therm. Anal. calorim., 113 (2013) 365-370.
[21] M. Moezzi, A. Ferencz, D. Lörinczy, Evaluation of blood plasma changes by differential scanning calorimetry in psoriatic patients treated with drugs, J. Therm. Anal. calorim., 116 (2014) 557-562.
[22] S. N. Rai, J. Pan, A. Cambon, J. B. Chaires, N. C. Garbett, Group Classification based on High-Dimensional Data: Application to Differential Scanning calorimetry Plasma Thermogram Analysis of Cervical Cancer and Control Samples, Open Access Medical Statistics, 3 (2013) 1-9.
[23] S. Todinova, S. Krumova, L. Gartcheva, C. Robeerst, S. G. Taneva, Microcalorimetry of blood serum proteome: a modified interaction network in the multiple myeloma case, Anal. Chem., 83 (2011) 7992-7998.
[24] S. Todinova, S. Krumova, P. Kurtev, V. Dimitrov, L. Djongov, Z. Dudunkov, S. G. Taneva, calorimetry-based profiling of blood plasma from colorectal cancer patients, Biochim. Biophys. Acta, 1820 (2012) 1879-1885.
[25] M. Wisniewski, N. C. Garbett, D. J. Fish, G. P. Brewood, J. J. Miller, J. B. Chaires, A. S. Benight, Differential Scanning calorimetry in Molecular Diagnostics, In Vitro Diagnostic Technology, 17 (2011) 29-34.
[26] I. Zapf, T. Fekecs, A. Ferencz, G. Tizedes, G. Pavlovics, E. Kãlmãn, D. Lörinczy, DSC analysis of human plasma in breast cancer patients, Thermochim. Acta, 524 (2011) 88-91.
[27] S. Vega, M. A. Garcia-Gonzalez, A. Lams, A. Velazquez-Campoy, O. Abian, Deconvolution Analysis for Classifying Gastric Adenocarcinoma Patients Based on Differential Scanning calorimetry Serum Thermograms, Sci. Rep., 5 (2015) 7988.
[28] L. Kikalishvili, M. Ramishvili, G. Nemsadze, T. Lezhava, P. Khorava, M. Gorgoshidze, M. Kiladze, J. Monaselidze, Thermal stability of blood plasma proteins of breast cancer patients, DSC study, J. Therm. Anal. calorim., 120 (2015) 501-505.
[29] I. Zapf, M. Moezzi, T. Fekecs, K. Nedvig, D. Lörinczy, A. Ferencz, Influence of oxidative injury and monitoring of blood plasma by DSC on breast cancer patients, J. Therm. Anal. calorim., (2015) DOI 10.1007/s10973-10015-14642-10979.
[30] S. Krumova, S. Todinova, A. Danailova, V. Petkova, K. Dimitrova, L. Gartcheva, S. G. Taneva, calorimetric features of IgM gammopathies. Implication for patient's diagnosis and monitoring, Thermochim. Acta, 615 (2015) 23-29.
[31] F. Barcelõ, J. J. Cerdà, A. Gutierrez, T. Jimenez-Marco, M. A. Durãn, A. Novo, T. Ros, A. Sampol, J. Portugal, Characterization of Monoclonal Gammopathy of Undetermined Significance by calorimetric Analysis of Blood Serum Proteome, PLOS ONE, 10 (2015) e0120316.
[32] M. Moezzi, I. Zapf, T. Fekecs, K. Nedvig, D. Lörinczy, A. Ferencz, Influence of oxidative injury and monitoring of blood plasma by DSC on patients with psoriasis, J. Therm. Anal. calorim., (2015) DOI 10.1007/s10973-10015-14674-10971.
[33] Z. Szalai, T. F. Molnar, D. Lörinczy, Differential scanning calorimetry (DSC) of blood serum in chronic obstructive pulmonary disease (COPD), J. Therm. Anal. calorim., 113 (2013) 259-264.
[34] A. Rasmussen, S. Sevier, J. A. Kelly, S. B. Glenn, T. Aberle, C. M. Cooney, A. Grether, E. James, J. Ning, J. Tesiram, J. Morrisey, T. Powe, M. Drexel, W. Daniel, B. Namjou, J. O. Ojwang, K. L. Nguyen, J. W. Cavett, J. L. Te, J. A. James, R. H. Scofield, K. Moser, G. S. Gilkeson, D. L. Kamen, C. W. Carson, A. I. Quintero-del-Rio, M. del Carmen Ballesteros, M. G. Punaro, D. R. Karp, D. J. Wallace, M. Weisman, J. T. Merrill, R. Rivera, M. A. Petri, D. A. Albert, L. R. Espinoza, T. O. Utset, T. S. Shaver, E. Arthur, J. M. Anaya, G. R. Bruner, J. B. Harley, The lupus family registry and repository, Rheumatology, 50 (2011) 47-59.
[35] M. C. Hochberg, Updating the American College of Rheumatology revised criteria for the classification of systemic lupus erythematosus, Arthritis Rheum, 40 (1997) 1725.
[36] T. Hastie, R. Tibshirani, J. Friedman, The Elements of Statistical Learning; Data Mining, Inference and Prediction, 2nd. Edition., Springer, New York, 2009.
[37] J. Ulbricht, lqa: Penalized Likelihood Inference for GLMs, R package version 1.0-3, 2012.
[38] J. Friedman, T. Hastie, R. Tibshirani, Regularization Paths for Generalized Linear Models via Coordinate Descent, J. Stat. Softw., 33 (2010) 1-22.
[39] V. Vapnik, The Nature of Statistical Learning Theory, Springer, New York, 1996.
[40] N. Becker, W. Werft, G. Toedt, P. Lichter, A. Benner, penalizedSVM: a R-package for feature selection SVM classification, Bioinformatics, 25 (2009) 1711-1712.
[41] N. Becker, G. Toedt, P. Lichter, A. Benner, Elastic SCAD as a novel penalization method for SVM classification tasks in high-dimensional data, BMC Bioinformatics, 12 (2011) 138.
[42] P. Xu, G. N. Brock, R. S. Parrish, Modified linear discriminant analysis approaches for classification of high-dimensional microarray data, Comput. Stat. Data Anal., 53 (2009) 1674-1687.
[43] D. M. Witten, R. Tibshirani, Penalized classification using Fisher's linear discriminant, J. R. Stat. Soc. Series B Stat. Methodol., 73 (2011) 753-772.
   [44] I. Gaynanova, J. G. Booth, M. T. Wells, Simultaneous sparse estimation of canonical vectors in the p»N setting, J. Am. Stat. Assoc., (2015) DOI: 10.1080/01621459.01622015.01034318.
[45] D. Witten, penalizedLDA: Penalized classification using Fisher's linear discriminant, R package version 1.0, 2011.
[46] I. Gaynanova, MGSDA: Multi-Group Sparse Discriminant Analysis, R package version 1.1, 2014.
[47] P. S. Gromski, H. Muhamadali, D. I. Ellis, Y. Xu, E. Correa, M. L. Turner, R. Goodacre, A tutorial review: Metabolomics and partial least squares-discriminant analysis—a marriage of convenience or a shotgun wedding, Anal. Chim. Acta, 879 (2015) 10-23.
[48] H. Chun, S. Keles, Sparse partial least squares for simultaneous dimension reduction and variable selection, J. R. Stat. Soc. Ser. B, 72 (2010) 3-25.
[49] D. Chung, S. Keles, Sparse partial least squares classification for high dimensional data, Stat. Appl. Genet. Mol. Biol., 9 (2010) Article 17.
[50] M. Kuhn, Building Predictive Models in R Using the caret Package, J. Stat. Softw., 28 (2008) 1-26. [51] Rivero S J, Diaz-Jouanen E, Alarcon-Segovia D. Lymphopenia in systemic lupus erythematosus.
Clinical, diagnostic, and prognostic significance. Arthritis Rheum 1978; 21:295-305.
[52] Rasmussen A, Sevier S, Kelly J A, Glenn S B, Aberle T, Cooney C M, et al. The lupus family registry and repository. Rheumatology 2011; 50:47-59.
[53] The Autoimmune Diseases Coordinating Committee. Progress in Autoimmune Diseases Research: National Institutes of Health, U.S. Department of Health and Human Services; 2005; NIH Publication No. 05-5140.
[54] Illei G G, Tackey E, Lapteva L, Lipsky P E. Biomarkers in Systemic Lupus Erythematosus: I I. Markers of Disease Activity. Arthritis Rheum 2004; 50:2048-65.
[55] Ahearn J M, Liu C-C, Kao A H, Manzi S. Biomarkers for systemic lupus erythematosus. Transl Res 2012; 159:326-42.
[56] Liu C-C, Manzi S, Kao A H, Navratil J S, Ahearn J M. Cell-Bound Complement Biomarkers for SLE: From Benchtop to Bedside. Rheum Dis Clin North Am 2010; 36:161-72.
[57] Kalunian K C, Chatham W W, Massarotti E M, Reyes-Thomas J, Harris C, Furie R A, et al. Measurement of cell-bound complement activation products enhances diagnostic performance in systemic lupus erythematosus. Arthritis Rheum 2012; 64:4040-7.
[58] Garbett N C, Miller J J, Jenson A B, Chaires J B. calorimetry outside the box: a new window into the plasma proteome. Biophys J 2008; 94:1377-83.
[59] Garbett N C, Mekmaysy C S, Helm C W, Jenson A B, Chaires J B. Differential scanning calorimetry of blood plasma for clinical diagnosis and monitoring. Exp Mol Pathol 2009; 86:186-91.
[60] Chagovetz A A, Jensen R L, Recht L, Glantz M, Chagovetz A M. Preliminary use of differential scanning calorimetry of cerebrospinal fluid for the diagnosis of glioblastoma multiforme. J Neurooncol 2011; 105:499-506.
[61] Chagovetz A A, Quinn C, Damarse N, Hansen L D, Chagovetz A M, Jensen R L. Differential scanning calorimetry of gliomas: a new tool in brain cancer diagnostics? Neurosurgery 2013; 73:289-95.

[62] Garbett N C, Merchant M L, Chaires J B, Klein J B. calorimetric analysis of the plasma proteome: Identification of type 1 diabetes patients with early renal function decline. Biochim Biophys Acta 2013; 1830:4675-80.
[63] Garbett N C, Merchant M L, Helm C W, Jenson A B, Klein J B, Chaires J B. Detection of Cervical Cancer Biomarker Patterns in Blood Plasma and Urine by Differential Scanning calorimetry and Mass Spectrometry. PLOS ONE 2014; 9:e84710. [64] Michnik A, Drzazga Z, Michalik K, Barczyk A, Santura I, Sozariska E, et al. Differential scanning calorimetry study of blood serum in chronic obstructive pulmonary disease. J Therm Anal calorim 2010; 102:57-60. [65] Todinova S, Krumova S, Kurtev P, Dimitrov V, Djongov L, Dudunkov Z, et al. calorimetry-based profiling of blood plasma from colorectal cancer patients. Biochim Biophys Acta 2012; 1820:1879-85. [66] Zapf I, Fekecs T, Ferencz A, Tizedes G, Pavlovics G, Kalman E, et al. DSC analysis of human plasma in breast cancer patients. Thermochim Acta 2011; 524:88-91. [67] Todinova S, Krumova S, Gartcheva L, Robeerst C, Taneva S G. Microcalorimetry of blood serum proteome: a modified interaction network in the multiple myeloma case. Anal Chem 2011; 83:7992-8. [68] Fekecs T, Zapf I, Ferencz A, Lörinczy D. Differential scanning calorimetry (DSC) analysis of human plasma in melanoma patients with or without regional lymph node metastases. J Therm Anal calorim 2012; 108:149-52. [69] Ferencz A, Fekecs T, Lörinczy D. Differential Scanning calorimetry, as a New Method to Monitor Human Plasma in Melanoma Patients with Regional Lymph Node or Distal Metastases. In: Xi Y (ed) Skin Cancer Overview. InTech, 2011, DOI: 10.5772/25606. Available: intechopen.com [70] Garbett N C, Miller J J, Jenson A B, Chaires J B. calorimetric Analysis of the Plasma Proteome.
Semin Nephrol 2007; 27:621-6. [71] Krumova S, Rukova B, Todinova S, Gartcheva L, Milanova V, Toncheva D, et al. calorimetric monitoring of the serum proteome in schizophrenia patients. Thermochim Acta 2013; 572:59-64.
[72] Mehdi M, Fekecs T, Zapf I, Ferencz A, Lörinczy D. Differential scanning calorimetry (DSC) analysis of human plasma in different psoriasis stages. J Therm Anal calorim 2013; 111:1801-4.
[73] Michnik A. Blood plasma, serum and serum proteins microcalorimetric studies aimed at diagnosis support. In: Lörinczy D (ed) Thermal Analysis in Medical Application. Akademiai Made′, 2011, 171-90.
[74] Fish D J, Brewood G P, Kim J S, Garbett N C, Chaires J B, Benight A S. Statistical analysis of plasma thermograms measured by differential scanning calorimetry. Biophys Chem 2010; 152:184-90.
[75] Rasmussen A, Sevier S, Kelly J A, Glenn S B, Aberle T, Cooney C M, et al. The lupus family registry and repository. Rheumatology 2011; 50:47-59.
[76] Hochberg M C. Updating the American College of Rheumatology revised criteria for the classification of systemic lupus erythematosus. Arthritis Rheum 1997; 40:1725.
[77] Benjamini Y, Hochberg Y. Controlling the false discovery rate: a practical and powerful approach to multiple testing. J R Stat Soc Ser B 1995; 57:289-300.
[78] Xu P, Brock G N, Parrish R S. Modified linear discriminant analysis approaches for classification of high-dimensional microarray data. Comput Stat Data Anal 2009; 53:1674-87.
[79] Garbett N C, Brock G N. Differential scanning calorimetry as a complementary diagnostic tool for the evaluation of biological samples. Biochim Biophys Acta 2016; 1860:981-9.
[80] Romero-Diaz J, Isenberg D, Ramsey-Goldman R. Measures of adult systemic lupus erythematosus: updated version of British Isles Lupus Assessment Group (BILAG 2004), European Consensus Lupus Activity Measurements (ECLAM), Systemic Lupus Activity Measure, Revised (SLAM-R), Systemic Lupus Activity Questionnaire for Population Studies (SLAQ), Systemic Lupus Erythematosus Disease Activity Index 2000 (SLEDAI-2K), and Systemic Lupus International Collaborating Clinics/American College of Rheumatology Damage Index (SDI). Arthritis Care Res 2011; 63:S37-46. [81] Petri M, Orbai A M, Alarcõn G S, Gordon C, Merrill J T, Fortin P R, et al. Derivation and validation of the Systemic Lupus International Collaborating Clinics classification criteria for systemic lupus erythematosus. Arthritis Rheum 2012; 64:2677-86.

[82] Ani\tilde{c} F, Zuvi\tilde{c}-Butorac M, Stimac D, Novak S. New classification criteria for systemic lupus erythematosus correlate with disease activity. Croat Med J 2014; 55:514-9. It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method of characterizing and/or predicting risk of a condition associated with biological sample obtained from a subject, comprising:
obtaining a thermal stability profile of the sample, using a sensor which detects heat capacity values;
applying a classification algorithm to the thermal stability profile; and
comparing the results to thermal stability data in the database to characterize and/or predict risk of the condition.

2. The method of claim 1, wherein the condition is systemic lupus erythematosus (SLE).

3. The method of claim 2, further comprising classifying the subject as having SLE when at least four of eleven ACR SLE criteria are present in the biological sample.

4. The method of claim 3, further comprising treating the subject for SLE.

5. The method of claim 1, wherein the classification algorithm is selected from one or more of logistic regression, support vector machines, Fisher's linear discriminant analysis, modified version of Fisher's linear discriminant analysis (MLDA), and partial least squares.

6. The method of claim 5, wherein the classification algorithm comprises a modified version of Fisher's linear discriminant analysis (MLDA).

7. The method of claim 6, further comprising a serological based classification, the MLDA and the serological based classification together providing increased sensitivity and overall accuracy for systemic lupus erythematosus (SLE) patients versus controls.

8. The method of claim 1, wherein the method characterizes the subject as having systemic lupus erythematosus (SLE) that is not detectable through antibody testing.

9. The method of claim 8, further comprising treating the subject for SLE.

10. The method of claim 1, wherein the subject is a female.

11. The method of claim 1, wherein the sensor comprises a differential scanning calorimeter (DSC).

12. The method of claim 1, further comprising characterizing the thermogram by one or more metrics selected from: (1) the total area under the thermogram (optionally from 45-90° C.); (2) the maximum excess specific heat capacity at various peaks; (3) the overall maximum peak height; (4) the width of the primary thermogram peak at half height; (5) the temperature of the peak maximum (Tₘₐₓ); (6) the ratio of the peak heights; and (7) the “mean” or first moment temperature of the thermogram, T_{FM}, where
$${T}_{FM} = \frac{{\int }_{45}^{90} ( {TC}_{p}^{ex} ) dT}{{\int }_{45}^{90} {C}_{p}^{ex} dT}$$
and C_{F}^{ex} represents the excess specific heat capacity at a given temperature.

13. A method of characterizing and/or predicting risk of a condition associated with biological sample obtained from a subject, comprising:
obtaining a thermogram of the sample, using a sensor which detects heat capacity values;
analyzing the thermogram using localized thermogram features and principal components; and
comparing the results to data in a database to characterize and/or predict risk of the condition.

14. The method of claim 13, further comprising applying a classification algorithm to the thermal stability profile.

15. The method of claim 13, wherein the condition is systemic lupus erythematosus (SLE).

16. The method of claim 15, further comprising classifying the subject as having SLE when at least four of eleven ACR SLE criteria are present in the biological sample.

17. The method of claim 15, further comprising treating the subject for SLE.

18. The method of claim 13, wherein the sensor comprises a differential scanning calorimeter (DSC).

19. The method of claim 13, further comprising characterizing the thermogram by one or more metrics selected from: (1) the total area under the thermogram (optionally from 45-90° C.); (2) the maximum excess specific heat capacity at various peaks; (3) the overall maximum peak height; (4) the width of the primary thermogram peak at half height; (5) the temperature of the peak maximum (Tₘₐₓ); (6) the ratio of the peak heights; and (7) the “mean” or first moment temperature of the thermogram, T_{FM}, where
$${T}_{FM} = \frac{{\int }_{45}^{90} ( {TC}_{p}^{ex} ) dT}{{\int }_{45}^{90} {C}_{p}^{ex} dT}$$
and C_{F}^{ex} represents the excess specific heat capacity at a given temperature.

20. A method of characterizing and/or predicting risk of a condition associated with biological sample obtained from a subject, comprising:
obtaining a thermogram of the sample, using a sensor which detects heat capacity values;
characterizing the thermogram by one or more metrics selected from: (1) the total area under the thermogram (optionally from 45-90° C.); (2) the maximum excess specific heat capacity at various peaks; (3) the overall maximum peak height; (4) the width of the primary thermogram peak at half height; (5) the temperature of the peak maximum (Tₘₐₓ); (6) the ratio of the peak heights; and (7) the “mean” or first moment temperature of the thermogram, T_{FM}, where
$${T}_{FM} = \frac{{\int }_{45}^{90} ( {TC}_{p}^{ex} ) dT}{{\int }_{45}^{90} {C}_{p}^{ex} dT}$$
and C_{F}^{ex} represents the excess specific heat capacity at a given temperature; and
comparing the results to data in a database to characterize and/or predict risk of the condition.
